(19) United States
(12) Patent Application Publication
(10) Pub. No.: US 2026/0338777 A1
(43) Pub. Date: Oct. 29, 2026

(54) COMPOSITIONS AND METHODS FOR MONITORING UBIQUITIN SIGNALING AND DEUBIQUITINASE ACTIVITY

(71) Applicant: The Texas A&M University System, College Station, TX (US)

(72) Inventors: Wenshe Ray Liu, College Station, TX (US); Saibal Chanda, College Station, TX (US)

(21) Appl. No.: 19/649,938

(22) Filed: Apr. 16, 2026

Related U.S. Application Data
(60) Provisional application No. 63/791,245, filed on Apr. 18, 2025.

(51) Int. Cl. G01N 33/573; G01N 33/58

(52) U.S. Cl. CPC G01N 33/573; G01N 33/58

(57) ABSTRACT

The present disclosure provides compositions and methods for monitoring enzyme activity. The disclosure further provides enzymatic probes, enzymatic fluorogenic substrates, and kits for monitoring ubiquitin and ubiquitin-like protein signaling and deubiquitinase and deubiquitinase-like enzyme activity.

### CROSS-REFERENCE TO RELATED APPLICATIONS

This application claims the priority of U.S. Provisional Appl. Ser. No. 63/791,245, filed Apr. 18, 2025, the entire disclosure of which is incorporated herein by reference.

### STATEMENT OF GOVERNMENT RIGHTS

This invention was made with government support under GM145351 awarded by the National Institute of Health. The government has certain rights in the invention. The development of this invention was funded in part by the Welch Foundation under grant number A-1715.

### FIELD OF THE INVENTION

The present disclosure relates to the field of biotechnology and more specifically to methods and compositions for detecting enzymatic activity, ubiquitin signaling, and deubiquitinase activity.

### BACKGROUND OF THE INVENTION

Ubiquitin is a small, evolutionarily conserved regulatory protein that is found in almost all eukaryotic organisms. It plays a crucial role in many cellular processes by attaching covalently to other proteins to regulate their function, location, or lifespan. This process is known as ubiquitination. Ubiquitination can lead to a variety of cellular outcomes. The most notable is to tag proteins for degradation by the proteasome. This mechanism of protein degradation, which is governed by the ubiquitin-proteasome system, is essential for the regulation of many fundamental cellular processes. Due to its crucial role in maintaining protein homeostasis, the ubiquitin-proteasome pathway has implications in the pathogenesis of various diseases. As such, it is a target for therapeutic intervention. As shown in FIG. 1, Panel A, protein ubiquitination operates through a highly regulated process involving a cascade of three groups of enzymes: E1 ubiquitin-activating enzymes, E2 ubiquitin-conjugating enzymes, and E3 ubiquitin ligases. Ubiquitination can stop at the attachment of a single ubiquitin (monoubiquitination) or continue to add a chain of ubiquitin molecules (polyubiquitination). This overall process is reversed by another group of enzymes, deubiquitinases (DUBs), that catalyze the hydrolysis of ubiquitin from a polyubiquitination chain or substrate. The present disclosure provides compositions and methods for activity-based protein profiling of cysteine enzymes in the ubiquitination/deubiquitination cascade.

### SUMMARY OF THE INVENTION

In one aspect, the present disclosure provides an enzymatic probe of formula I:

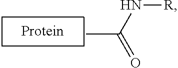

wherein the protein is ubiquitin, a ubiquitin-like protein, or a variant thereof, and wherein R is a hydrazinecarboxylate ester. In another aspect, the present disclosure provides an enzymatic fluorogenic substrate of formula II:

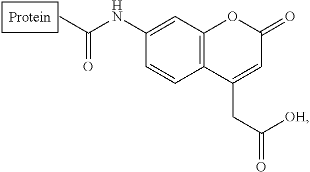 [SMILES: CC(=O)Nc1ccc2c(CC(=O)O)cc(=O)oc2c1]

wherein the protein is ubiquitin, a ubiquitin-like protein, or a variant thereof.

Ubiquitin-like proteins include, but are not limited to, SUMO1, SUMO2, SUMO3, SUMO4, URM1, UFM1, UFM2, NEDD8, ISG15, FAT10, GABARAP, GABARAPL1, GABARAPL2, MAP1LC3A, MAP1LC3B, MAP1LC3C, ATG12, UBL5, MNSFβ, TUBL, BUBL1, FUBI, and NUB1L. In one embodiment, a variant may comprise a C-terminal deletion of at least one amino acid. In another embodiment, a variant may comprise a deletion of a C-terminal glycine. In yet another embodiment, a variant may be ubiquitin₁₋₇₅ or ubiquitin₁₋₇₄. Non-limiting examples of hydrazinecarboxylates include methyl hydrazinecarboxylate, ethyl hydrazinecarboxylate, 2,2,2-trifluoroethyl-2-glycylhydrazine-1-carboxylate, vinyl hydrazinecarboxylate, allyl hydrazinecarboxylate, and phenyl hydrazinecarboxylate.

In one embodiment, an enzymatic probe, an enzymatic fluorogenic substrate, or a composition of the present disclosure may further comprise a detectable label. Detectable labels include, but are not limited to, a protein tag, a Flag tag, a hemagglutinin A tag, a myc tag, a polyhistidine tag, a CBP tag, a GST tag, an HBH tag, a MBP tag, a S tag, a Sumo tag, a V5 tag, biotin, concanavalin A, a fluorescent label, a chromophore, an avi-tag, and a radioactive label. In another embodiment, the protein of an enzymatic probe of the present disclosure may be further defined as an azapeptide. In yet another embodiment, the protein of an enzymatic fluorogenic substrate of the present disclosure may be further defined as an amide conjugate. In yet another embodiment, the ubiquitin, the ubiquitin-like protein, or the variant thereof of an enzymatic fluorogenic substrate of the present disclosure comprises a C-terminal glycine-to-cysteine mutation.

In certain embodiments, the present disclosure provides a kit comprising an enzymatic probe or an enzymatic fluorogenic substrate of the present disclosure. In one embodiment, a kit of the present disclosure may comprise at least 2 or at least 3 enzymatic probes of formula I. in another embodiment, a kit may comprise at least two enzymatic fluorogenic substrates, wherein each of the enzymatic fluorogenic substrates comprises a different ubiquitin-like protein or a variant thereof.

In yet another aspect, the present disclosure provides a method for determining enzymatic activity comprising a) contacting a sample comprising at least one enzyme with an enzymatic probe of the present disclosure; and b) detecting formation of a covalent complex comprising the enzyme and the enzymatic probe, wherein the formation of the covalent complex indicates the presence of enzymatic activity. In one embodiment, the enzyme is a cysteine enzyme. Non-limiting examples of a cysteine enzyme include a USP family enzyme, a UCH family enzyme, a Josephin family enzyme, an OTU family enzyme, a ZUFSP family enzyme, an E1 enzyme, an E2 enzyme, a cysteine-containing E3 enzyme, USP5, USP2, USP7, USP8, USP10, USP14, USP15, USP16, USP18, USP25, UFSP2, CYLD, UCHL1, UCHL3, UCHL5, Ataxin-3, JOSD2, OTUB1, OTUB2, OTULIN, OTUD6B, Cezanne-OTUD7B, OTUD2, OTUD7C, UBE1, UBE2E2, Parkin, UBE3A, ITCH-E3A, USP9X, and USP21. In another embodiment, a sample may comprise a plurality of enzymes. In yet another embodiment, a method of the present disclosure may comprise contacting a sample with at least 2 or at least 3 enzymatic probes of formula I.

In yet another aspect, the present disclosure provides a method of determining enzymatic activity comprising a) contacting a sample comprising at least one enzyme with an enzymatic fluorogenic substrate of the present disclosure; and b) detecting hydrolysis of the enzymatic fluorogenic substrate, wherein hydrolysis of the enzymatic fluorogenic substrate indicates the presence of enzymatic activity. In one embodiment, detecting hydrolysis comprises detecting a fluorescent signal or detecting release of the detectable label. In another embodiment, the enzyme is a deubiquitinase or deubiquitinase-like enzyme. Non-limiting examples of deubiquitinase and deubiquitinase-like enzymes include a USP family enzyme, a UCH family enzyme, a Josephin family enzyme, an OTU family enzyme, a ZUFSP family enzyme, a JAMM/MPN+ family enzyme, a MINDY family enzyme, a metalloenzyme, USP5, USP2, USP7, USP8, USP10, USP14, USP15, USP16, USP18, USP25, UFSP2, CYLD, UCHL1, UCHL3, UCHL5, Ataxin-3, JOSD2, OTUB1, OTUB2, OTULIN, OTUD6B, Cezanne-OTUD7B, OTUD2, OTUD7C, SENP1, ATG4B, USP9X, and USP21.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows the ubiquitination/deubiquitination cascade and ubiquitin probes. Panel A—The E1-E2-E3 enzymatic cascade to covalently attach ubiquitin (Ub) to a protein substrate (Sub) and the reverse deubiquitination process catalyzed by deubiquitinase (DUB). Both E3 and DUB enzymes have two classes, either with a catalytic cysteine or without. Cysteine-based DUBs form covalent ubiquitin complexes only transiently that quickly hydrolyze. Panel B—Ubiquitin probes that have been developed so far for the study of enzymes in the ubiquitination/deubiquitination cascade. A ubiquitinated protein model is shown as a comparison. The ubiquitin Gly76 Cα and carboxyl carbon atoms are labeled as 1 and 2, respectively. The arrow pointing at 2 in Ub-Sub indicates the hydrolysis position that is also the site transiently conjugating with a catalytic DUB cysteine. Sites in a variety of probes that can covalently conjugate with cysteine enzymes in the ubiquitination/deubiquitination cascade are indicated with pointing arrows. Panel C—Ubiquitin azapeptide esters that mimic a ubiquitinated protein (Ub-Sub) better than conventional ubiquitin probes for covalent conjugation with DUBs.

FIG. 2 shows the activated cysteine-based protein ligation (ACPL) technique and its use in the synthesis of ubiquitin azapeptide ester probes. Panel A—Chemical mechanism of ACPL. A protein cysteine is activated by a nitrile donor molecule, e.g., 2-nitro-5-thiocyanatobenzoic acid (NTCB) to form a 1-acyl-2-iminothiazolidine intermediate that undergoes nucleophilic acyl substitution with an amine to generate a C-terminally functionalized protein. Panel B—Use of two ubiquitin proteins, Flag-Ub₁₋₇₅-Cys-6×His and Flag-Ub₁₋₇₄-Cys-6×His, to react with four amines including propargylamine (Pa), methyl hydrazinecarboxylate (MTC), ethyl hydrazinecarboxylate (ETC), and 2,2,2-trifluoroethyl-2-glycylhydrazine-1-carboxylate (TFEGHC), mediated by NTCB, to generate four ubiquitin azapeptide ester probes including FlagUbPa, FlagUbMTC, FlagUbETC, and FlagUbTFEGHC. Panel C—Deconvoluted electrospray ionization mass spectrometry (ESI-MS) spectra of four synthesized ubiquitin probes. Determined molecular weights match well with their calculated values. Panel D—Covalent conjugation of four ubiquitin probes with USP5. The generated USP5-Ub complexes are highlighted. Bovine serum albumin (BSA) was used as a loading control.

FIG. 3 demonstrates that ubiquitin azapeptide esters are covalent probes for cysteine-containing DUBs. DUBs were reacted with four probes at 37 °C for 40 min before they were analyzed by SDS-PAGE and probed by an anti-FLAG antibody. Covalent DUB-Ub complexes are indicated. BSA was used as a loading control for all reactions. Panel A—USP family DUBs including (i) USP2, (ii) USP7, (iii) USP8, (iv) USP10, (v) USP14, (vi) USP15, (vii) USP25, and (viii) CYLD. Panel B—A UCH family DUB-UCHL3. Panel C—Josephin family DUBs (i) Ataxin-3 and (ii) JOSD2. Panel D—OTU family DUBs including (i) OTUB2, (ii) OTULIN, (iii) Cezanne-OTUD7B, (iv) OTUD2, and (v) OTUD7C.

FIG. 4 demonstrates covalent complex formation between three Ub azapeptide probes and E1, E2 and cysteine-containing E3 enzymes. Panel A—UBE1. Panel B—UbE2E2. Panel C—Parkin. Panel D—UbE3A. Panel E—ITCH. The probes and the enzymes were reconstituted in a reaction buffer containing BSA. The BSA was detected using anti-BSA antibody. Since, BSA is not related to the ubiquitination/deubiquitination cascade and does not have any cross-reactivity, it was used as a loading control for the reaction.

FIG. 5 shows the characterization of a USP7-Ub thiocarbamate complex. Panel A—Structure of a USP7-Ub thiocarbamate complex generated from FlagUbTFEGHC and USP7 CD. Panel B—Time dependence of the complex formation at 37 °C. Panel C—Stability of the complex at different temperatures for 60 min. Panel D—Stability of the complex over time at 37 °C. Panel E—Stability of the complex in the presence of varying concentrations of glutathione for 60 min. Panel F—Iodoacetamide blocks the complex formation.

FIG. 6 shows activity-based protein profiling using three ubiquitin azapeptide ester probes. Panel A—Cysteine enzymes profiled from HEK293T cell lysates using FlagUbPa and three ubiquitin azapeptide ester probes. Cell lysates were reacted with four probes for 20 h before their SDS-PAGE analysis and Western blotting by anti-FLAG. Panel B—Cysteine enzymes from HEK293T cells profiled specifically by three ubiquitin azapeptide ester probes. Cell lysates were first reacted with UbPa for 12 h to allow UbPa-reacting enzymes to bind and then treated with FlagUbPa and three ubiquitin azapeptide ester probes for an additional 12 h before their SDS-PAGE analysis and Western blotting by anti-FLAG. Enzymes that were uniquely labeled by ubiquitin azapeptide ester probes are indicated. Panel C—Cross-interaction network of identified cysteine enzymes from samples in Panel B. Panel D—Venn diagram illustrated the distribution of the same enzymes presented in Panel C. Panels E, F, G, H, I, J—Western blot validation of UCHL1, UCHL3, UCHL5, UbE2D3, OTUB1, and OTUD6B in anti-FLAG immunoprecipitated samples from HEK293T cell lysates treated with UbPa for 12 h and then three ubiquitin azapeptide esters for additional 12 h. Final Western blot detection was completed using corresponding antibodies for the 6 named enzymes.

FIG. 7 shows covalent complex formation between the Ub azapeptide ester probes and ataxin-3 in HEK293T cell lysate. HEK293T cell lysates were reacted with four probes for 20 hrs at 4 °C before Western blotting analyses by an anti-ataxin-3 antibody. Enzyme-probe complexes that are unique to three ubiquitin azapeptide ester probes are indicated. Unlike the azapeptide ester probes, no covalent complex formation was observed for FlagUbPa.

FIG. 8 shows the covalent labeling trajectories of 5 DUBs, including (Panel A) UCHL1, (Panel B) UCHL5, (Panel C) UCHL3, (Panel D) OTUB1, and (Panel E) OTUD6B with four synthetic ubiquitin probes analyzed by anti-Flag-based Western blot analyses. UCHL1 and UCHL5 showed far quicker reaction kinetics with three ubiquitin azapeptide ester probes than with FlagUbPa.

FIG. 9 shows the characterization of four synthetic ubiquitin probes on their inhibition of DUB-catalyzed fluorescent 7-amino-4-coumarinyl-acetic acid (ACA) release from fluorogenic UbACA. Panel A—Structure of UbACA. Panel B—Catalytic release of ACA from UbACA by UCHL1, UCHL5, and USP25. 2 μM UbACA was incubated with 20 nM DUB 20 nM, and fluorescence increase (Ex: 360 nM; Em: 460) was then monitored. Panel C, Panel D, and Panel E—Inhibition curves of UCHL1, UCHL5, and USP25 by four synthetic ubiquitin probes characterized by enzyme activity on UbACA. 20 nM DUB was used to catalyze ACA release from 2 μM UbACA in the presence of various concentrations of a ubiquitin probe. The initial 5-min reaction velocities for ACA release were used to plot inhibition curves.

FIG. 10 shows the profiling of cysteine enzymes in the ubiquitination/deubiquitination cascade in different mouse tissues including kidney (Panel A), brain (Panel B), liver (Panel C), and lung (Panel D) by four synthetic ubiquitin probes and profiling the same tissues, kidney (Panel E), brain (Panel F), liver (Panel G), and lung (Panel H), with three ubiquitin azapeptide ester probes after their reaction with UbPa to remove UbPa-reacting enzymes. For all tissues, there are bands clearly visible for three ubiquitin azapeptide ester probes but not for FlagUbPa.

FIG. 11 shows time-dependent fluorescence increase upon enzymatic cleavage of Ub-ACA by UCHL1, UCHL3, UCHL5, USP2, USP5, USP7, USP9X, USP15, and USP21.

FIG. 12 shows time-dependent fluorescence increase upon enzymatic cleavage of FLAG-SUMO1-4-ACA substrates by SENP1.

FIG. 13 shows time-dependent fluorescence increase upon enzymatic cleavage of FLAG-NEDD8-ACA by UCHL3, SENP8, and USP21, and the absence of cleavage by USP5 (negative control).

FIG. 14 shows time-dependent fluorescence increase upon enzymatic cleavage of FLAG-GABARAP-ACA and FLAG-GABARAPL2 by ATG4B.

FIG. 15 shows time-dependent fluorescence increase upon enzymatic cleavage of FLAG-ISG15-ACA by USP18 and FLAG-UFM1-ACA by UFSP2.

FIG. 16 shows time-dependent fluorescence increase upon enzymatic cleavage of FLAG-MNSFβ-ACA by USP16.

### DETAILED DESCRIPTION OF THE INVENTION

The present disclosure provides compositions and methods for monitoring enzymatic activity, ubiquitin and ubiquitin-like protein signaling, and deubiquitinase activity. The present disclosure provides a significant advance in the art by providing highly sensitive and specific probes and enzymatic fluorogenic substrates for measuring enzymatic activity. In one embodiment, the present disclosure provides ubiquitin and ubiquitin-like azapeptide esters as optimal activity-based probes for mapping the activity of cysteine enzymes.

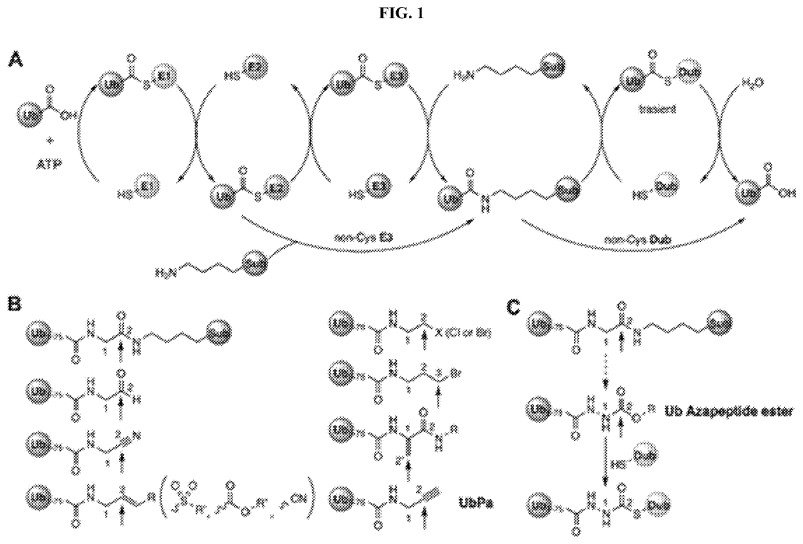

Ubiquitin is a small, evolutionarily conserved regulatory protein that is found in almost all eukaryotic organisms. It plays a crucial role in many cellular processes by attaching covalently to other proteins to regulate their function, location, or lifespan. This process is known as ubiquitination. Ubiquitination can lead to a variety of cellular outcomes. The most notable is to tag proteins for degradation by the proteasome. This mechanism, the ubiquitin-proteasome system, is essential for the regulation of many fundamental cellular processes. Due to its crucial role in maintaining protein homeostasis, the ubiquitin-proteasome pathway has implications in the pathogenesis of various diseases. As such, it is a target for therapeutic intervention. As shown in FIG. 1, Panel A, protein ubiquitination operates through a highly regulated process involving a cascade of three groups of enzymes: E1 ubiquitin-activating enzymes, E2 ubiquitin-conjugating enzymes, and E3 ubiquitin ligases. Ubiquitination can stop at the attachment of a single ubiquitin (monoubiquitination) or continue to add a chain of ubiquitin molecules (polyubiquitination). This overall process is reversed by another group of enzymes, deubiquitinases (DUBs), that catalyze the hydrolysis of ubiquitin from a polyubiquitination chain or substrate. Existing ubiquitin and ubiquitin-like protein probes often fail to accurately mimic natural enzyme-substrate interactions, affecting the comprehensiveness of enzyme profiling. The present disclosure provides compositions and methods for activity-based protein profiling of cysteine enzymes, such as those in the ubiquitination/deubiquitination and ubiquitin-like protein signaling cascades that overcome these limitations. For example, the enzymatic probes provided by the present disclosure closely mimic a native ubiquitin or ubiquitin-like protein substrate and react with cysteine enzymes through a mechanism akin to natural enzymatic catalysis. As a result, the compositions and methods of the present disclosure facilitate the identification of enzymes that require strict substrate binding and/or have an active site that doesn't accommodate the transition state intermediate formed with conventional ubiquitin and ubiquitin-like probes.

Furthermore, the enzymatic fluorogenic substrates of the present disclosure provide a significant advance in the art as these enzymatic fluorogenic substrates are water soluble and can be synthesized in the lab, significantly reducing the costs compared to the current, expensive commercial enzymatic fluorogenic substrates. With their improved solubility, lower cost, and excellent performance in enzyme studies, the enzyme substrates of the present disclosure are superior tools for researchers studying deubiquitinases, drug discovery, and enzyme activity.

In some aspects, the methods and compositions of the present disclosure may be useful for biochemical assays, enzyme profiling, inhibitor screening, high-throughput analysis, and diagnostic assays for diseases associated with ubiquitin or ubiquitin-like protein dysregulation, such as cancer, neurodegenerative disease, and immune disorders. Furthermore, the distinct enzyme labeling patterns observed in different tissues using the enzymatic probes of the present disclosure demonstrate application in biomarker identification and personalized medicine.

### A. Enzymatic Probes and Enzymatic Fluorogenic Substrates

In certain aspects, the present disclosure provides an enzymatic probe of formula I:

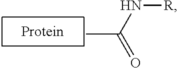

wherein the protein is ubiquitin, a ubiquitin-like protein, or a variant thereof, and wherein R is a hydrazinecarboxylate ester. Hydrazinecarboxylates are known in the art and any such hydrazinecarboxylate may be used according to certain embodiments of the present disclosure. Non-limiting examples of hydrazinecarboxylates include methyl hydrazinecarboxylate, ethyl hydrazinecarboxylate, 2,2,2-trifluoroethyl-2-glycylhydrazine-1-carboxylate, vinyl hydrazinecarboxylate, allyl hydrazinecarboxylate, and phenyl hydrazinecarboxylate. As used herein, the term “ester” refers to a chemical compound derived from an acid in which at least one —OH hydroxyl group is replaced by an —O— alkyl (alkoxy) group. As used herein the term “ubiquitin-like protein” refers to a protein that is involved in the posttranslational modification of various macromolecules. In certain embodiments, a ubiquitin-like protein may comprise a β-grasp fold similar to the β-grasp fold of ubiquitin. Ubiquitin-like proteins are involved in a diverse set of cellular processes, including but not limited to nuclear transport, proteolysis, translation, autophagy, and antiviral pathways. Ubiquitin-like proteins are known in the art and any such ubiquitin-like protein may be used according to certain embodiments of the present disclosure. Ubiquitin-like proteins include, but are not limited to, small ubiquitin-like modifier 1 (SUMO1), small ubiquitin-like modifier 2 (SUMO2), small ubiquitin-like modifier 3 (SUMO3), small ubiquitin-like modifier 4 (SUMO4), ubiquitin-related modifier 1 (URM1), ubiquitin fold modifier 1 (UFM1), ubiquitin fold modifier 2 (UFM2), NEDD8 ubiquitin like modifier (NEDD8), interferon-stimulated gene 15 (ISG15), human leukocyte antigen F-associated transcript 10 (FAT10), gamma-aminobutyric acid receptor-associated protein (GABARAP), GABA type A receptor associated protein like 1 (GABARAPL1), GABA type A receptor associated protein like 2 (GABARAPL2), microtubule-associated protein 1 light chain 3 alpha (MAP1LC3A), microtubule associated protein 1 light chain 3 beta (MAP1LC3B), microtubule-associated protein 1 light chain 3 gamma (MAP1LC3C), autophagy related 12 (ATG12), monoclonal non-specific suppressor factor β (MNSFβ), TINCR-encoded ubiquitin-like protein (TUBL), BIL-ubiquitin-like 1 (BUBL1), FUBI, and NEDD8 ultimate buster 1 long (NUBIL).

SUMO1, SUMO2, SUMO3, and SUMO4 are members of the small ubiquitin-like modifier protein family. These proteins function similar to ubiquitin in that they are bound to target proteins as part of a post-translational modification system. Unlike ubiquitin, which targets proteins for degradation, the SUMO proteins are involved in a variety of cellular processes, such as nuclear transport, transcriptional regulation, apoptosis, and protein stability.

URM1 functions as a sulfur carrier in tRNA thiolation by means of a mechanism that requires the formation of a thiocarboxylate at the C-terminal glycine residue of URM1. URM1 also modifies proteins in the yeast ubiquitin-like urmylation pathway.

UFM1 is a ubiquitin-like protein that is conjugated to target proteins by E1-like activating enzymes, such as UBA5, and E2-like conjugating enzyme, such as UFC1, in a manner analogous to ubiquitylation.

UFM2 is a ubiquitin-like protein that is conjugated to target proteins in a manner analogous to ubiquitylation.

NEDD8 enables ubiquitin protein ligase binding activity. Neddylation is a process by which the ubiquitin-like protein NEDD8 is conjugated to target proteins. This process is similar to ubiquitination but relies on different pathway enzymes. Neddylation often causes structural changes in target proteins.

ISG15 is a ubiquitin-like protein that is conjugated to intracellular target proteins upon activation by interferon-alpha and interferon-beta. ISG15 provides functions such as chemotactic activity towards neutrophils, direction of ligated target proteins to intermediate filaments, cell-to-cell signaling, and antiviral activity during viral infections.

FAT10, also known as ubiquitin D, acts similarly to ubiquitin by covalently modifying proteins and tagging them for destruction by the proteasome.

GABA receptors are ligand-gated chloride channels that mediate inhibitory neurotransmission. GABARAP is highly positively charged in its N-terminus and clusters neurotransmitter receptors by mediating interaction with the cytoskeleton.

GABARAPL1 enables Tat protein binding activity, phospholipid binding activity, and ubiquitin protein ligase binding activity. GABARAPL1 increases cell surface expression of kappa-type opioid receptors by facilitating anterograde intracellular trafficking of the receptor and is involved in the formation of autophagosomal vacuoles.

GABARAPL2 enables phosphatidylethanolamine binding activity and ubiquitin protein ligase binding activity. GABARAPL2 is involved in the negative regulation of the proteasomal protein catabolic process and protein localization in the endoplasmic reticulum.

MAP1LC3A, MAP1LC3B, and MAP1LC3C enable microtubule binding activity, phosphatidylethanolamine binding activity, ubiquitin protein ligase binding activity, autophagy, and/or membrane trafficking.

ATG12 is a ubiquitin-like protein involved in autophagy vesicle formation.

MNSFβ is a ubiquitin-like family member protein that is involved in various biological functions. MNSFβ covalently binds to intracellular pro-apoptotic protein BCl-G and regulates the extracellular-signal related kinase (ERK)/mitogen-activated protein kinase (MAPK) cascade.

TUBL is a ubiquitin-like protein that regulates cell cycle progression and cellular protein levels.

BUBL1 comprises a bacterial intein-like (BIL domain), which stimulates an N—O or N—S acyl rearrangement at the boundary with its upstream sequences and to catalyze cleavage and self-splicing reactions. BIL domains in the BUBL proteins also trigger such (N—S) acyl shifts and catalyze nucleophilic attack on the resulting thioester by a protein during autocatalytic processing, leading to ligation of the upstream Ubl sequence to the attacking molecule. If the attacking group is a Lys side chain of a protein, the resulting product would be a Ubl-protein conjugate similar to those formed through the ubiquitination pathway.

FUBI is a ubiquitin-like protein, which has an N-terminal ubiquitin-like domain fused to the ribosomal protein S30. The FUBI domain acts as a substitute or an inhibitor of ubiquitin, FAT10, or NEDD8.

NUB1L suppresses atypical neddylation and promotes the proteasomal degradation of misfolded proteins.

As used herein the term “variant” when used in reference to a protein or polypeptide refers to a modified version of a protein or polypeptide. As is known in the art, amino acid residues may be changed in a polypeptide sequence to create an equivalent, or even improved, second-generation variant polypeptide. For example, certain amino acids may be substituted for other amino acids in a polypeptide sequence without appreciable loss of function, binding capacity, or binding specificity. Since the binding capacity, the binding specificity, and the nature of a protein define the functional activity of a protein, certain amino acid substitutions can be made in a protein sequence, and/or in its corresponding DNA coding sequence, such that the resultant variant protein comprises similar or desirable properties of the original protein. Thus, in some embodiments, the polynucleotide and polypeptide sequences of the present disclosure may comprise various amino acid or nucleic acid substitutions, deletions, and/or insertions without appreciable loss of biological utility or activity. As used herein the term “functionally equivalent codon” refers to codons that encode the same amino acid, such as the six different codons known in the art which code for arginine. As used herein, the terms “neutral substitutions” and “neutral mutations” refer to a change in a polypeptide sequence, or the encoding nucleotide sequence, such that the sequence comprises or encodes a biologically equivalent amino acid compared to that found in the original sequence. In certain embodiments, any amino acid of any polypeptide described herein may be substituted with any biologically equivalent amino acid. Biologically equivalent amino acids are known in the art.

Nucleic acid or amino acid sequence variants of the disclosure may comprise, in some embodiments, a substitution, an insertion, or a deletion. A polypeptide variant of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the polypeptide, as compared to the referenced polypeptide or to the wild-type polypeptide, including any range derivable therebetween. A variant polypeptide may comprise, for example, an amino acid sequence having at least 50%, 60%, 70%, 80%, 90%, 95%, 96%, 97%, 98%, or 99% or more sequence identity to the wild type sequence of a protein selected from the group consisting of SUMO1, SUMO2, SUMO3, SUMO4, URM1, UFM1, UFM2, NEDD8, ISG15, FAT10, GABARAP, GABARAPL1, GABARAPL2, MAP1LC3A, MAP1LC3B, MAP1LC3C, ATG12, UBL5, MNSFβ, TUBL, BUBL1, FUBI, and NUB1L. A polypeptide may include, for example, at least 1, 2, 3, 4, 5, 6, 7, 8, 9, 10, 11, 12, 13, 14, 15, 16, 17, 18, 19, or 20 amino acid substitutions. In certain embodiments, a variant polypeptide of the present disclosure may comprise a deletion of at least 1, at least 2, at least 3, at least 4, or at least 5 C-terminal or N-terminal amino acids. In one embodiment, a variant polypeptide may be ubiquitin₁₋₇₅, which comprises amino acids 1-75 of ubiquitin or ubiquitin₁₋₇₄, which comprises amino acids 1-74 of ubiquitin. In another embodiment, a variant polypeptide may comprise a deletion of one or more C-terminal glycine(s). As is known in the art a number of ubiquitin and ubiquitin-like proteins either naturally comprise a C-terminal glycine or are processed to expose a C-terminal glycine. Non-limiting examples of such proteins are shown in Table 1.

TABLE 1
Ubiquitin and Ubiquitin-like Proteins
Comprising a C-terminal Glycine.

| Protein | C-terminal Motif (after processing) |
|---|---|
| Ubiquitin | Gly-Gly (G76) |
| SUMO1-4 | Gly-Gly |
| NEDD8 | Gly-Gly |
| ISG15 | Gly-Gly |
| FAT10 | Gly-Gly |
| UFM1 | Gly |
| ATG12 | Gly |
| URM1 | Gly |
| GABARAP | Gly |
| GABARAPL1 | Gly |
| GABARAPL2 | Gly |
| MAP1LC3A | Gly |
| MAP1LC3B | Gly |
| UBL5 | Gly-Gly |
| MNSFβ | Gly |
| FUBI | Gly |
| NUB1L | Gly |
| TUBL | Gly (predicted) |
| BUBL1 | Gly (predicted) |

It also will be understood that amino acid and nucleic acid sequences may include additional residues, such as additional N- or C-terminal amino acids, or 5′ or 3′ sequences, respectively, and yet still be essentially identical to the sequences provided by the present disclosure. Such essentially identical sequences, in some embodiments, may maintain the biological activity described herein for the sequences of the present disclosure. The addition of terminal sequences particularly applies to nucleic acid sequences that may, for example, include various non-coding sequences flanking either of the 5′ or 3′ portions of the coding region.

Deletion variants typically lack one or more amino acid residues compared to the protein from which the variant was derived, the native protein, or the wild-type protein. In certain embodiments, individual amino acid residues may be deleted, or a number of contiguous amino acids may be deleted. In one embodiment, a stop codon may be introduced, for example by substitution or insertion, into an encoding nucleic acid sequence to generate a truncated protein variant.

Insertional variants typically involve the addition of one or more amino acid residues at a non-terminal point of a polypeptide. Terminal additions may also be generated and can include fusion proteins. Non-limiting examples of such fusion proteins include multimers or concatemers of one or more polypeptides provided by the present disclosure.

Substitutional variants typically comprise the exchange of one amino acid for another at one or more sites within the polypeptide. Substitutional variants may be designed to modulate one or more properties of the polypeptide, with or without the loss of other functions or properties of the polypeptide. Amino acid substitutions may be conservative amino acid substitutions. As used herein, the term “conservative amino acid substitution” refers to an amino acid substitution wherein one amino acid is replaced with another amino acid having similar chemical properties. Conservative amino acid substitutions may involve, for example, the exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may, in some embodiments, encompass non-naturally occurring amino acid residues, which are typically incorporated by chemical peptide synthesis rather than by synthesis in biological systems. These include peptidomimetics or other reversed or inverted forms of amino acid moieties.

One skilled in the art can determine suitable polypeptide variants, as set forth herein, using well-known techniques. For example, one skilled in the art may identify suitable areas of the polypeptide molecule that may be changed without affecting activity by targeting regions not believed to be critical for activity. The skilled artisan will also be able to identify amino acid residues and portions of the polypeptide molecules that are conserved among similar proteins or polypeptides. In some embodiments, regions of a polypeptide molecule that may be important for biological activity or for structure may be subject to conservative amino acid substitutions without significantly altering the biological activity or adversely affecting the protein structure.

When making conservative or non-conservative amino acid substitutions, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. These values are isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain aspects, the substitution of amino acids whose hydropathy indices are within ±2 is included. In some aspects of the disclosure, those that are within ±1 are included, and in other aspects of the disclosure, those within ±0.5 are included.

As is known in the art, the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain aspects, the greatest local average hydrophilicity of a protein, as governed by the hydrophilicity of its adjacent amino acids, correlates with the binding capacity, binding specificity, and biological function of a protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In some embodiments, amino acid substitutions based upon similar hydrophilicity values may include the substitution of amino acids whose hydrophilicity values are within ±2 of each other. In one embodiment, the substitution of amino acids whose hydrophilicity values are within ±1 or within ±0.5 are included. As is known in the art, an amino acid can be substituted for another having a similar hydrophilicity value and still produce a biologically equivalent protein.

Additionally, it is well within the knowledge of a person of ordinary skill in the art to review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such amino acid residues predicted to be important.

One skilled in the art can also analyze the three-dimensional structure of a protein along with the amino acid sequence in relation to that structure in similar proteins. In view of such information, one skilled in the art may predict the alignment of amino acid residues of a protein with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments. This information may allow one skilled in the art to determine the amino acid positions where further substitutions should be avoided either alone or in combination with other substitutions. Various tools available to determine secondary structure can be found on the world wide web, for example, at expasy.org/proteomic s/protein structure.

Among the four groups of enzymes in the ubiquitination/deubiquitination cascade, E1 and E2 enzymes are restrictedly cysteine enzymes, a small number of E3 enzymes are cysteine enzymes, and most DUB enzymes are cysteine proteases. All cysteine enzymes in this pathway form a covalent ubiquitin complex, either stable or transient, during their catalysis. This catalytic nature and the high nucleophilicity of their catalytic cysteine have led to the development of a variety of ubiquitin-based covalent probes for these enzymes, as shown in FIG. 1, Panel B.

Covalent ubiquitin probes may be used, in some embodiments, to conduct activity-based protein profiling of cysteine enzymes in the ubiquitination/deubiquitination cascade. Activity-based protein profiling is a powerful technique to study activities of a group of enzymes within complex biological systems. It is particularly useful for identifying active enzymes in a given sample. Applying this technique in combination with the use of different covalent ubiquitin probes has led to the elucidation of functionally active cysteine enzymes, mainly DUBs, in the ubiquitin cascade pathway in both tissues and disease cells. However, conventional ubiquitin probes, as shown in FIG. 1, Panel B, are structurally different from a native ubiquitinated protein. Except for ubiquitin aldehyde that forms a thiohemiacetal adduct with an enzyme active site cysteine to mimic the enzyme's transition state, all other probes react with the catalytic cysteine in enzymes through mechanisms different from the enzymes' catalytic mechanisms. Ubiquitin aldehyde cannot be used as an activity-based probe. Using the previously available probes in activity-based protein profiling analysis does not allow for the identification of cysteine enzymes in the ubiquitination/deubiquitination cascade that require strict substrate binding and/or have an active site that doesn't accommodate the transition state intermediate formed with conventional ubiquitin probes. The present disclosure, in certain embodiments, provides ubiquitin azapeptide esters, non-limiting examples of which are shown in FIG. 1C, as optimal activity-based probes for cysteine enzymes in the ubiquitination/deubiquitination cascade.

In certain embodiments, a protein of the present disclosure may be further defined as an azapeptide. As used herein, the term “azapeptide” refers to a peptide molecule containing a nitrogen atom named as aza-nitrogen, in an original Cα position in the peptide backbone. This modification alters the electronic configuration of the peptide. An “azapeptide ester” as used herein refers to an azapeptide that also has the C-terminal side of its aza-nitrogen converted to an ester. An azapeptide ester is structurally highly similar to its parent peptide and therefore maintains a similar binding pattern to a target enzyme. In addition, the ester as part of a carbamate group is chemically stable but reactive toward cysteine proteases to form covalent adducts. Carbamate in an azapeptide ester has a more electron deficient carbon that facilitates reaction toward a nucleophile compared to an original amide bond. Comparatively, the alkyne group in the conventional probe UbPa is relatively inert toward a nucleophile in physiological conditions. It has been shown that this covalent adduct is stable when the aza-amino acid is azaglycine, although other aza-amino acids will lead to a slowly hydrolyzing adduct. Since ubiquitin naturally has a C-terminal glycine, ubiquitin azapeptide esters, as shown in FIG. 1, Panel C, with their C-terminal glycine converted to an azaglycine ester structurally and functionally mimic a ubiquitinated protein. These proteins therefore react with cysteine-based DUB enzymes to form a stable covalent adduct that is a thiocarbamate. This azaglycine modification retains backbone flexibility similar to glycine, ensuring that a probe can still fit well within the narrow catalytic cleft of the enzymes. A ubiquitin azapeptide ester reacts with a DUB active site cysteine in a similar manner as a ubiquitinated protein substrate to form a tetrahedral transition state, typical for acyl transfer reactions, which has an oxygen anion stabilized by the anion hole in the enzyme. Due to its better chemical reactivity toward a nucleophile than the alkyne in UbPa, azapeptide ester in a ubiquitin probe is more potent toward some DUBs. Due to its highly reactive nature toward an activated cysteine in an enzyme, ubiquitin azapeptide esters are also active toward E1, E2, and cysteine-containing E3 enzymes, mimicking their substrates. Therefore, a ubiquitin azapeptide ester may be applied as an activity-based probe to profile cysteine enzymes in the ubiquitination/deubiquitination cascade. The reactivity of a ubiquitin azapeptide ester may also be tuned through the use of different leaving alcohol groups. This is easily achievable synthetically and provides an additional advantage.

In particular embodiments, an enzymatic probe of the present disclosure may be useful for determining enzymatic activity. In one embodiment, the present disclosure provides a method comprising a) contacting a sample comprising at least one enzyme with an enzymatic probe of the present disclosure; and b) detecting formation of a covalent complex comprising the enzyme and the enzymatic probe, wherein the formation of the covalent complex indicates the presence of enzymatic activity. In certain embodiments, the enzyme may be a cysteine enzyme. As used herein the term “cysteine enzyme” refers to an enzyme comprising at least one catalytic site cysteine. Any cysteine enzyme known in the art may be useful in certain embodiments of the present disclosure. Non-limiting examples of cysteine enzymes include a USP family enzyme, a UCH family enzyme, a Josephin family enzyme, an OTU family enzyme, an E1 enzyme, an E2 enzyme, a cysteine-containing E3 enzyme, USP5, USP2, USP7, USP8, USP10, USP14, USP15, USP16, USP18, USP25, UFSP2, CYLD, UCHL1, UCHL3, UCHL5, Ataxin-3, JOSD2, OTUB1, OTUB2, OTULIN, OTUD6B, Cezanne-OTUD7B, OTUD2, OTUD7C, UBE1, UBE2E2, Parkin, UBE3A, ITCH-E3A, USP9X, and USP21. In certain embodiments, an enzymatic probe of the present disclosure may be used to determine the enzymatic activity of a plurality of enzymes comprised within a sample, such as at least 2, at least 3, at least 4, at least 5, at least 10, at least 15, at least 20, at least 25, at least 30, at least 35, at least 40, at least 45, or at least 50 enzymes, including all ranges and values derivable therebetween. A method of the present disclosure may comprise, in particular embodiments, contacting a sample with at least 2, at least 3, at least 4, or at least 5 enzymatic probes of the present disclosure. In one embodiment, each of the at least 2, at least 3, at least 4, or at least 5 enzymatic probes have a different structure.

In particular aspects, the present disclosure provides an enzymatic fluorogenic substrate of formula II:

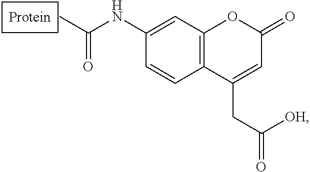 [SMILES: CC(=O)Nc1ccc2c(CC(=O)O)cc(=O)oc2c1]

wherein the protein is ubiquitin, a ubiquitin-like protein, or a variant thereof. In one embodiment, the ubiquitin-like protein is selected from the group consisting of SUMO1, SUMO2, SUMO3, SUMO4, URM1, UFM1, NEDD8, ISG15, FAT10, GABARAP, GABARAPL1, GABARAPL2, MAP1LC3A, MAP1LC3B, MAP1LC3C, ATG12, UBL5, MNSFβ, TUBL, BUBL1, FUBI, and NUB1L. In another embodiment, the protein is further defined as an amide conjugate. The enzymatic fluorogenic substrates of the present disclosure provide a significant advance in the art over the commonly used ubiquitin-7-amino-4-methylcoumarin (Ub-AMC). Ub-AMC suffers from poor solubility in both water and organic solvents, limiting its effectiveness in biochemical assays and high-throughput screening. This results in inconsistent data and reduced sensitivity in enzyme studies. Additionally, Ub-AMC is highly expensive to produce, even at microgram scales, making it impractical for routine research use. In contrast the enzymatic fluorogenic substrates of the present disclosure are water soluble and can be synthesized in the lab, significantly reducing the costs compared to expensive commercial Ub-AMC. With their improved solubility, lower cost, and excellent performance in enzyme studies, the enzyme substrates of the present disclosure are superior tools for researchers studying deubiquitinases, drug discovery, and enzyme activity.

In certain aspects, an enzymatic fluorogenic substrate of the present disclosure may be useful for determining enzymatic activity. In one embodiment, the present disclosure provides a method comprising a) contacting a sample comprising at least one enzyme with an enzymatic fluorogenic substrate as described herein; and b) detecting hydrolysis of the enzymatic fluorogenic substrate, wherein hydrolysis of the enzymatic fluorogenic substrate indicates the presence of enzymatic activity. In certain embodiments, hydrolysis may be detected by detecting a fluorescent signal. In one embodiment, the enzyme may be a deubiquitinase or a deubiquitinase-like protein. As used herein the term “deubiquitinase-like protein” refers to an enzyme that functions to remove ubiquitin or a ubiquitin-like protein from its substrate. Any deubiquitinase or deubiquitinase-like protein known in the art may be used according to particular embodiments of the present disclosure. Non-limiting examples of deubiquitinases include a USP family enzyme, a UCH family enzyme, a Josephin family enzyme, an OTU family enzyme, a ZUFSP family enzyme, a JAMM/MPN+ family enzyme, a MINDY family enzyme, a metalloenzyme, USP5, USP2, USP7, USP8, USP10, USP14, USP15, USP16, USP18, USP25, UFSP2, CYLD, UCHL1, UCHL3, UCHL5, Ataxin-3, JOSD2, OTUB1, OTUB2, OTULIN, OTUD6B, Cezanne-OTUD7B, OTUD2, OTUD7C, SENP1, ATG4B, USP9X, and USP21.

As used herein the term “sample” refers to a material or mixture of materials containing at least one component of interest. As used herein the term “biological sample” refers to a sample obtained from a biological subject. A biological sample of the present disclosure may include, but is not limited to, a cell sample, a tissue sample, a tumor sample, a body fluid sample, a blood sample, a urine sample, a feces sample, a semen sample, a serum sample, a plasma sample, a saliva sample, and a cerebrospinal fluid sample.

### B. Detectable Labels

In certain aspects, the present disclosure provides compositions and methods for detection and labeling of the enzymatic probes and enzymatic fluorogenic substrates of the present disclosure. Methods for labeling and detection of polypeptide molecules are well-known in the art, and any such method known in the art may be used to label or detect the polypeptides described herein. As a non-limiting example, polypeptides may be labeled with a detectable moiety, such as a protein tag, a radioactive atom, a chromophore, a fluorophore, or the like, and then detected using methods known in the art. Such labeled polypeptides may be used, in some embodiments, for in vivo or in vitro diagnostic techniques.

As used herein, the term “label” refers to a directly or indirectly detectable compound or composition that is conjugated directly or indirectly to the composition to be detected. In certain embodiments, a polynucleotide molecule, protein, or cell may be labeled to generate a labeled composition. In some embodiments, labeled compositions include sequences that are conjugated to a protein tag. As used herein, the term “protein tag” refers to a detectable peptide sequence that has been fused to a protein of interest. Non-limiting examples of protein tags include an affinity tag, a chromatography tag, an epitope tag, a fluorescent tag, a Flag tag, a hemagglutinin A tag, a myc tag, a polyhistidine tag, a CBP tag, a GST tag, an HBH tag, a MBP tag, a S tag, a Sumo tag, a V5 tag, biotin, an avi-tag, and concanavalin A. A label may be detectable by itself (e.g., radioisotope labels or fluorescent labels) or, in the case of an enzymatic label, may catalyze chemical alteration of a substrate compound or composition that is detectable. Labels may be suitable for small scale detection or for high-throughput screening. As such, suitable labels include, but are not limited to radioisotopes, fluorochromes, chemiluminescent compounds, dyes, and proteins, including enzymes. Labels may be simply detected or may be quantified. In certain embodiments, labels that may be quantified provide a numerically reportable value. In luminescence or fluorescence assays, the detectable response may be generated directly using a luminophore or fluorophore associated with an assay component involved in binding, or indirectly using a luminophore or fluorophore associated with another (e.g., reporter or indicator) component.

### C. Kits

In certain aspects, the present disclosure provides a kit comprising an enzymatic probe or an enzymatic fluorogenic substrate of the present disclosure. In certain embodiments, a kit of the present disclosure may comprise at least 2, at least 3, at least 4, or at least 5 enzymatic probes of formula I. Each of the at least 2, at least 3, at least 4, or at least 5 enzymatic probes of the kit, in one embodiment, may comprise a different structure of formula I. In another embodiment, a kit of the present disclosure may comprise at least 2, at least 3, at least 4, or at least 5 enzymatic fluorogenic substrates of formula II. Each of the at least 2, at least 3, at least 4, or at least 5 enzymatic fluorogenic substrates of the kit, in one embodiment, may comprise a different structure of formula II. A kit of the present disclosure may, in certain embodiments, include components for any suitable assay platform that may be used to determine ubiquitin or ubiquitin-like protein signaling or deubiquitinase activity. A kit of the present disclosure may include, in certain embodiments, a dipstick, a membrane, a chip, a disk, a test strip, a filter, a microsphere, a slide, a multi-well plate, an optical fiber, or a solid support system for capturing protein complexes. Examples of solid support systems include, but are not limited to, plastic, silicon, metal, resin, glass, membrane, gel, polymer, sheet, polysaccharide, capillary, film, plate, and slide solid support systems. In one embodiment, the kit may further comprise instructions for use.

The term “about” is used to indicate that a value includes the standard deviation of the mean for the device or method being employed to determine the value. The use of the term “or” in the claims is used to mean “and/or” unless explicitly indicated to refer to alternatives only or the alternatives are mutually exclusive. When used in conjunction with the word “comprising” or other open language in the claims, the words “a” and “an” denote “one or more,” unless specifically noted otherwise. The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps. Similarly, any system or method that “comprises,” “has,” or “includes” one or more components is not limited to possessing only those components and covers other unlisted components.

Other objects, features, and advantages of the present disclosure are apparent from the detailed description provided herein. It should be understood, however, that the detailed description and any specific examples provided, while indicating specific embodiments of the disclosure, are given by way of illustration only, since various changes and modifications within the spirit and scope of the disclosure will become apparent to those skilled in the art from this detailed description. Any embodiment of the present disclosure may be used in combination with any other embodiment described herein.

All references are incorporated herein by reference in their entirety.

### EXAMPLES

The following examples are included to illustrate embodiments of the present disclosure. It should be appreciated by those of skill in the art that the techniques disclosed in the examples that follow represent techniques discovered by the inventor to function well in the practice of the invention. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1: Synthesis of FlagUbPa and Ubiquitin Azapeptide Esters

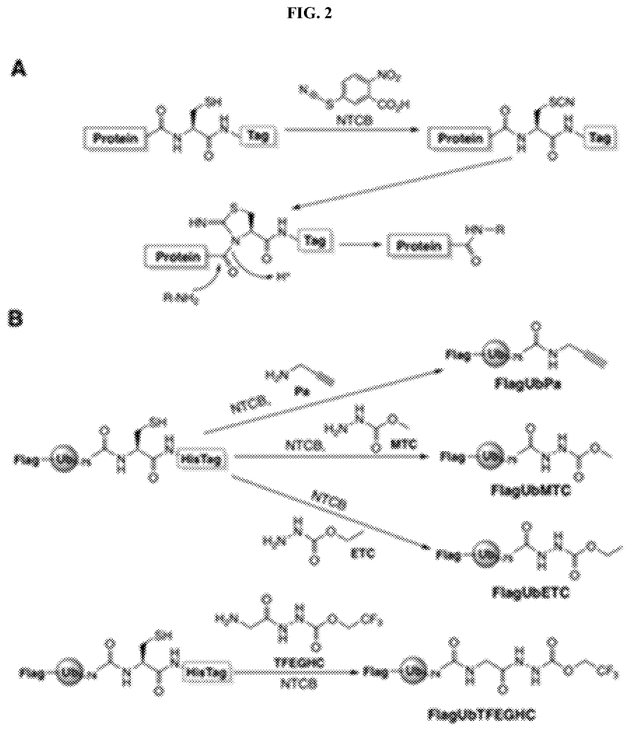

The expressed protein ligation technique, in which a protein with intein fused at its C-terminus is recombinantly produced to form a protein thioester, has been most commonly used to generate a ubiquitin thioester for a subsequent reaction with a small molecule to afford a conventional ubiquitin probe. An alternative expressed protein ligation technique that requires no involvement of intein or any enzymes has also been developed and applied to the synthesis of ubiquitin and ubiquitin-like protein-small molecule conjugates. This technique, coined as activated cysteine-based protein ligation (ACPL), features a nitrile donor molecule, e.g., 2-nitro-5-thiocyanobenzoic acid (NTCB), to activate a protein cysteine as shown in FIG. 2, Panel A to form a 1-acyl-2-iminothiazolidine intermediate that subsequently undergoes a nucleophilic acyl substitution reaction with a small molecule amine. The overall reaction leads to replacing cysteine with the amine at the C-terminus of the target protein. Ubiquitin is naturally devoid of cysteine, however, a large variety of small molecule amines including different amino acids can be conjugated to the ubiquitin C-terminus using a recombinant approach to install a cysteine residue at the C-terminus and then performing an ACPL reaction of the purified protein. As demonstrated by the present disclosure, an ACPL may also be used to generate ubiquitin azapeptide esters. Two recombinant ubiquitin proteins were expressed and purified from E. coli. The first is Flag-Ub₁₋₇₅-Cys-6×His which has a G76C mutation followed by a 6×His tag and the second Flag-Ub₁₋₇₄-Cys-6×His which contains a G75C mutation followed by a 6×His tag. Both proteins have an N-terminal FLAG tag epitope for immunoprecipitation and Western blot analysis using an anti-FLAG antibody. Flag-Ub₁₋₇₅-Pa (FlagUbPa) was synthesized as a control probe using the ACPL protocol described in Yu et. al., Methods Mol. Biol. 2022, 2530, 159-167 by incubating Flag-Ub₁₋₇₅-Cys-6×His with tris(2-carboxyethyl)phosphine (TCEP), NTCB, and propargylamine. FlagUbPa was used during the whole study as a control molecule due to its wide use as an activity-based probe for DUB enzymes. Embodiments of ubiquitin azapeptide esters that can serve as probes for cysteine enzymes in the ubiquitination/deubiquitination cascade include, but are not limited to, Ub₁₋₇₅ azaglycine esters. They can be made from Flag-Ub₁₋₇₅-Cys-6×His by reacting it with different hydrazinecarboxylates. To tune reactivity in the final ubiquitin azapeptide esters, three probes were initially designed with methanol, ethanol, and trifluoroethanol leaving groups, respectively. As such, three hydrazinecarboxylates, methyl hydrazinecarboxylate (MTC), ethyl hydrazinecarboxylate (ETC), and 2,2,2-trifluoroethyl hydrazinecarboxylate were either acquired from commercial providers or synthesized for their reactions with Flag-Ub₁₋₇₅-Cys-6×His in the presence of TCEP and NTCB (FIG. 2 Panel B). Using MTC and ETC led to successful synthesis of Flag-Ub₁₋₇₅-MTC (FlagUbMTC) and Flag-Ub₁₋₇₅-ETC (FlagUbETC), respectively. However, 2,2,2-trifluoroethyl hydrazinecarboxylate has low solubility in water, leading to a low reaction yield. As such, 2,2,2-trifluoroethyl-2-glycylhydrazine-1-carboxylate (TFEGHC) was synthesized. TFEGHC is soluble in water. Reacting it with Flag-Ub₁₋₇₄-Cys-6×His in the presence of TCEP and NTCB afforded Flag-Ub₁₋₇₄-TFEGHC (FlagUbTFEGHC) (FIG. 2, Panel B). Note that the three ubiquitin azapeptide probes, FlagUbMTC, FlagUbETC, and FlagUbTFEGHC, differ only at their C-terminal alcohol leaving group. FlagUbPa and the three developed ubiquitin azapeptide probes were isolated using fast protein liquid chromatography (FPLC) to high purity and then analyzed by electrospray ionization mass spectrometry (ESI-MS). The two original proteins, Flag-Ub₁₋₇₅-Cys-6×His and Flag-Ub₁₋₇₄-Cys-6×His were analyzed by ESI-MS as well for quality control. Original proton-charge and deconvoluted spectra were determined for all proteins. The deconvoluted ESI-MS spectra for the four probes FlagUbPa, FlagUbMTC, FlagUbETC, and FlagUbTFEGHC with their determined molecular weights indicated are shown in FIG. 2, Panel C. For all probes, their determined molecular weights match well with their theoretical values with a deviation of 0.3 Da, confirming their successful synthesis.

### Example 2: Demonstrations of Ubiquitin Azapeptide Esters as Covalent Probes for Cysteine-Containing DUBs in the Ubiquitination/Deubiquitination Cascade

Three ubiquitin azapeptide esters were analyzed for use as covalent probes for cysteine enzymes in the ubiquitination/deubiquitination cascade. USP5 is a commercially available DUB enzyme that has a catalytic cysteine. This enzyme was used in reactions with FlagUbPa and three ubiquitin azapeptide esters. USP5 was incubated with the probes for 40 min at 37 °C and then analyzed by SDS-PAGE analysis followed by Western blotting with anti-FLAG. The result is presented in FIG. 2, Panel D.

USP5 itself has no FLAG tag, showing a blank lane in the Western blot image. On the contrary, its reactions with four probes led to the formation of a USP5-ubiquitin complex with a molecular weight above 100 kDa that was clearly detected by anti-FLAG. To ensure equal loading of both enzymes and probes, all reactions were conducted in a DUB reaction buffer containing bovine serum albumin (BSA) as a loading control that was detected using an anti-BSA antibody. For USP5, FlagUbMTC and FlagUbETC displayed reactivity like FlagUbPa. FlagUbTFEGHC showed weaker reactivity than the other three. Compared to methyl and ethyl groups in FlagUbMTC and FlagUbETC, respectively, the trifluoroethyl group in FlagUbTFEGHC is more electron-withdrawing. Therefore, chemically FlagUbTFEGHC is more reactive than FlagUbMTC and FlagUbETC. Despite the different reactivities that were observed, the results clearly demonstrated that all three ubiquitin azapeptide esters can serve as activity-based probes for cysteine-containing DUB enzymes.

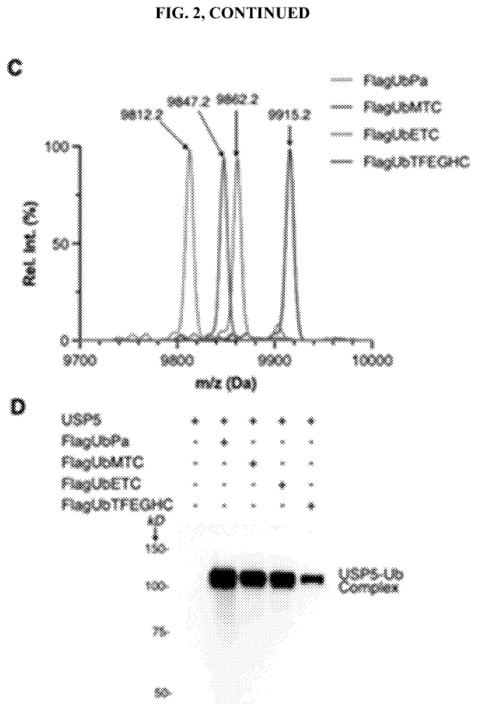

Investigations were expanded to evaluate the reactivity of three ubiquitin azapeptide esters across a diverse set of DUBs, all of which feature an active site cysteine. Sixteen DUBs, including eight from the USP family (USP2, USP7, USP8, USP10, USP14, USP15, USP16, USP18, USP25, and CYLD), one from the UCH family (UCHL3), two from the Josephin family (Ataxin-3 and JOSD2), and five from the OTU family (OTUB2, OTULIN, Cezanne-OTUD7B, OTUD2, and OTUD7C) were tested with the three ubiquitin azapeptide esters, with FlagUbPa serving as a control. Reactions of all DUBs with FlagUbPa and three ubiquitin azapeptide ester probes were conducted and analyzed in the same manner as for USP5. As shown in FIG. 3, Panels A-D, the tested DUB enzymes exhibited distinct reactivity profiles toward four probes. USP2 and UCHL3 showed comparable reactivity with all probes, displaying strong labeling efficiency (FIG. 3, Panel A(i) and Panel B). In contrast, USP7 demonstrated marked selectivity for FlagUbTFEGHC although overall labeling efficiency with all probes was strong (FIG. 3, Panel A (ii)). The azapeptide ester probes exhibited higher reactivity compared to FlagUbPa with USP8, USP10, and USP25 (FIG. 3, Panel A (iii), (iv), and (vii)). Both FlagUbMTC and FlagUbPa showed comparable reactivity toward USP14 (FIG. 3, Panel A (v)), while FlagUbETC and FlagUbTFEGHC showed reduced reactivity. USP15 exhibited robust labeling with all four probes, with FlagUbPa being slightly more efficient than the three azapeptide ester probes (FIG. 3, Panel A (vi)). FlagUbPa and FlagUbTFEGHC exhibited similar reactivity toward CYLD, compared to relatively weaker reactivity for FlagUbMTC and FlagUbETC toward this enzyme (FIG. 3, Panel A (viii)). In the Josephin family, Ataxin-3 displayed a strong preference for ubiquitin azapeptide esters, whereas FlagUbPa exhibited minimal reactivity (FIG. 3, Panel C (i)). For JOSD2, FlagUbMTC and FlagUbETC demonstrated covalent labeling comparable to FlagUbPa, while FlagUbTFEGHC showed reduced labeling (FIG. 3, Panel C (ii)). The OTU family DUBs presented diverse covalent labeling patterns with four probes. FlagUbMTC and FlagUbETC exhibited enhanced reactivity with OTUB2 compared to FlagUbPa and FlagUbTFEGHC (FIG. 3, Panel D(i)), while FlagUbMTC demonstrated superior labeling of OTULIN (FIG. 3, Panel D (ii)), outperforming all other probes and indicating it as an optimal ligand for this enzyme. Notably, FlagUbPa showed almost no reactivity toward OTULIN. For Cezanne-OTUD7B, all probes labeled the enzyme with similar high efficiency (FIG. 3, Panel D (iii)). OTUD2 showed strong labeling with azapeptide ester probes, particularly with FlagUbMTC, while FlagUbPa displayed weak reactivity (FIG. 3, Panel D (iv)). Interestingly, FlagUbPa failed to label OTUD7C, as indicated by the absence of a covalent complex band, where two of the azapeptide ester probes successfully engaged with the enzyme, underscoring a potential advantage of ubiquitin azapeptide esters for enzymes with stringent substrate specificity (FIG. 3, Panel D (v)). OTU DUBs often display isopeptide linkage selectivity during their catalyzed reactions. This higher reaction selectivity requirement likely contributes to their relatively better reactivity toward ubiquitin azapeptide ester probes.

To rule out nonspecific covalent labeling of DUBs caused solely by the azapeptide ester group, a short peptide, biotin-GG-LRG-MTC was synthesized, which retained the C-terminal three residues of ubiquitin and azaglycine. This peptide was used to label all DUBs tested in FIG. 2 and FIG. 3. The reactions were conducted under identical conditions, with the only modification being the substitution of a ubiquitin probe with biotin-GG-LRG-MTC. Due to the peptide's low molecular weight (<1 kDa), the reaction mixtures were incubated with streptavidin before being analyzed by SDS-PAGE and probed using an anti-streptavidin antibody via Western blotting. No covalent complex formation was observed between biotin-GG-LRG-MTC and any of the tested DUBs. These results demonstrate that, in the absence of the full ubiquitin scaffold to enhance binding, the azapeptide ester functional group alone is insufficient to induce covalent labeling of DUBs.

The collected data demonstrates that azapeptide ester probes are generally better probes than FlagUbPa for covalent conjugation with DUBs. Except USP15 that displayed strong labeling with all probes among which FlagUbPa was slightly better, all tested DUBs exhibited reactivity comparable to or greater than FlagUbPa when tested with at least one azapeptide ester probe. Notably, certain OTU DUBs that were not labeled by FlagUbPa were effectively labeled by the azapeptide ester probes. OTU DUBs are known to recognize isopeptide linkages for more stringent substrate selectivity than USP DUBs. For OTULIN, OTUD2, and OTUD7C, their low to no labeling with FlagUbPa but efficient labeling with the ubiquitin azapeptide ester probes is likely due to the azapeptide ester probes' resemblance to native ubiquitinated protein substrates. Low labeling of Ataxin 3 with FlagUbPa is likely due to the same reason. Therefore, applying ubiquitin azapeptide ester probes for activity-based protein profiling reveals cysteine enzymes that are not targeted by FlagUbPa.

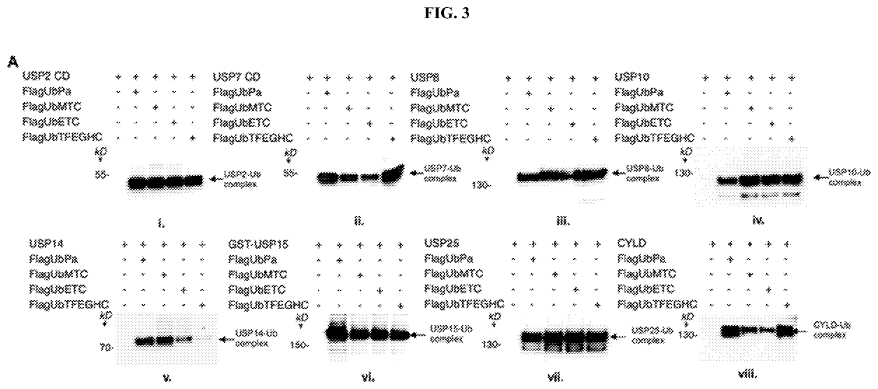

A selected group of E1, E2, and cysteine-containing E3 enzymes including UBE1, UBE2E2, Parkin, UBE3A, and ITCH-E3A were used to react with the three ubiquitin azapeptide ester probes. As shown in FIG. 4, these enzymes reacted with the ubiquitin azapeptide ester probes with different preferences, demonstrating that ubiquitin azapeptide ester probes also serve as activity-based probes for E1, E2, and cysteine-containing E3.

### Example 3: Formation and Stability of a DUB-Ub Thiocarbamate Covalent Complex

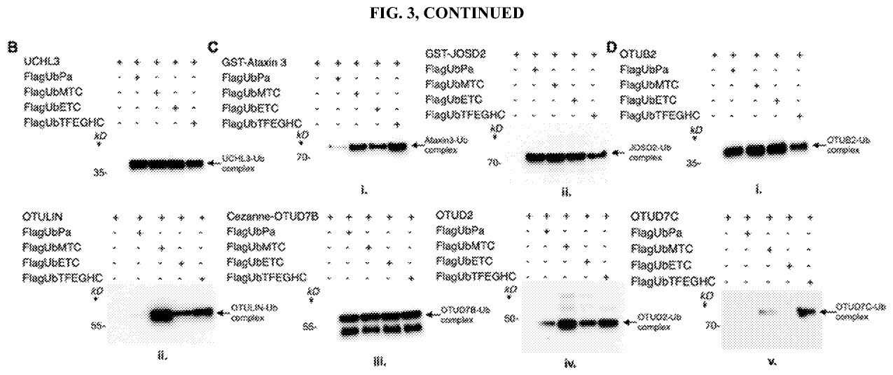

As shown in FIG. 3, USP7 exhibited highly efficient and specific labeling with all three ubiquitin azapeptide ester probes. Therefore, USP7 was selected as a model to demonstrate formation and stability of a covalent DUB complex formed with a ubiquitin azapeptide ester probe. To synthesize the USP7-Ub covalent complex (FIG. 5, Panel A), USP7 CD was incubated with FlagUbTFEGHC and BSA used as a loading control. To assess whether complex formation increased over time, a time-dependent assay was performed by incubating USP7 CD with FlagUbTFEGHC for a total reaction time of 40 min at 37 °C. At designated time points (0*[operation dead time<30 s], 5, 10, 20, and 40 min), the reaction was quenched by adding SDS-PAGE loading dye and subsequently analyzed. As shown in FIG. 5, Panel B, the reaction reached completion by approximately 5 min. To evaluate the thermal stability, the USP7-Ub covalent complex was subjected to different temperatures (37 °C, 45 °C, 50 °C, 55 °C, and 60 °C) for 60 min. As shown in FIG. 5, Panel C, the complex remained stable up to 45 °C but began to decompose at 50 °C and above. Next, the stability of the USP7-Ub covalent complex was evaluated over an extended period. The complex was incubated at 37 °C for up to 24 h. As shown in FIG. 5, Panel D, the complex remained stable throughout the entire duration. Additionally, the stability of the thiocarbamate bond was examined in the presence of varying concentrations of glutathione using a thiol-thioester exchange assay conducted over 60 min. As shown in FIG. 5, Panel E, except for a slight decrease in the Western blot signal at 10 mM glutathione, the complex remained stable across all tested concentrations. To confirm that formation of the USP7-Ub covalent complex requires an intact active-site cysteine, USP7 CD was pre-treated with 10 mM iodoacetamide before incubation with FlagUbTFEGHC. As shown in FIG. 5, Panel F, iodoacetamide treatment completely inhibited complex formation, confirming that the USP7-Ub covalent complex was formed via a thiocarbamate linkage at the active-site cysteine. The results demonstrate that ubiquitin azapeptide probes form stable complexes with cysteine-containing enzymes through a thiocarbamate linkage. This stability, both over time and under varying conditions, supports their viability as effective activity-based probes for cysteine enzymes in the ubiquitination/deubiquitination cascade.

### Example 4: Activity-Based Protein Profiling of Cysteine Enzymes in the Ubiquitination/Deubiquitination Cascade Using Ubiquitin Azapeptide Ester Probes

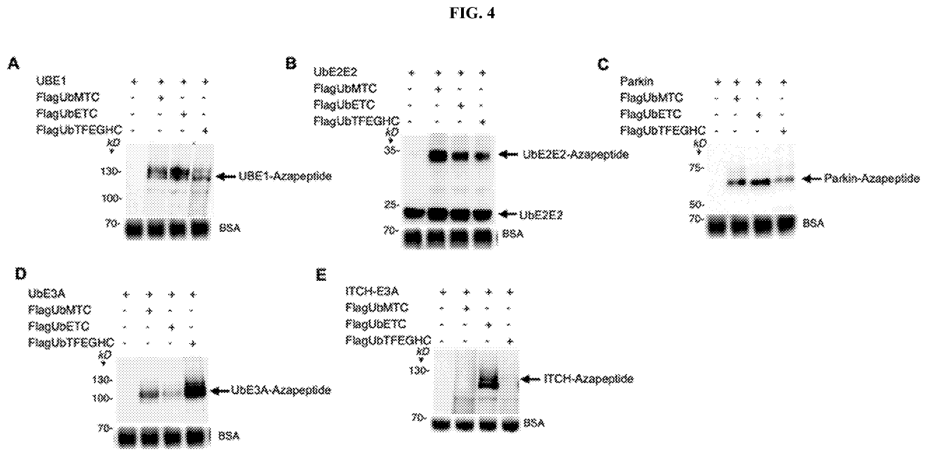

To assess the ability of ubiquitin azapeptide esters in mapping cysteine enzyme activities in the ubiquitination/deubiquitination cascade, labeling experiments of HEK293T cell lysates with three ubiquitin azapeptide esters were performed. FlagUbPa was used as a control. To simplify the analysis, only the lysate supernatant was collected and used to react with four probes and the same cell lysate samples were used for all four probes for every group of experiments to avoid potential variations between different batches of HEK293T cell lysates. The clarified supernatant was incubated with each of the four probes for 20 h at 4 °C before analyzing the reaction mixtures by SDS-PAGE and Western blot analysis with anti-FLAG. The result is shown in FIG. 6, Panel A. Without adding a probe, there was only one visible band around 75 kDa detected by anti-FLAG, confirming anti-FLAG as a clean antibody for the activity-based protein profiling analysis. Reacting HEK293T cell lysates with all four probes led to the formation of a slew of enzyme-ubiquitin conjugates that were strongly detected by anti-FLAG. The four labeled samples clearly showed multiple similarly positioning bands in the Western blot gel that likely resulted from the same cysteine enzymes. However, there are bands that were uniquely present in the ubiquitin azapeptide ester-labeled samples but weakly or not detectable in the FlagUbPa-labeled sample. For example, there was an exceptionally strong Western blot band at 37 kDa for all three azapeptide ester probe-treated samples. Although the FlagUbPa-treated sample has a band at a similar position, it is significantly weaker, and it is difficult to assess whether this is from another enzyme. A negative control was also conducted with Flag-Ub₁₋₇₅-Cys-6×His. Incubating HEK293T cell lysates with Flag-Ub₁₋₇₅-Cys-6×His did not result in any significant labeling of proteins, confirming that chemical reactivity at the C-terminus of FlagUbPa and the three ubiquitin azapeptide ester probes toward an activated cysteine in an enzyme is required for profiling cysteine enzymes in the ubiquitination/deubiquitination cascade.

To further explore cysteine enzymes that may be uniquely labeled with ubiquitin azapeptide esters, cysteine enzymes from HEK293T cell lysates that were pretreated first with UbPa to inactivate UbPa-reactive enzymes were profiled with three ubiquitin azapeptide ester probes. UbPa lacks a FLAG tag to avoid Western blot detection by anti-FLAG. To synthesize UbPa, Ub₁₋₇₅-Cys-6×His was expressed, purified, and then used to synthesize UbPa by incubation with TCEP, NTCB, and propargylamine. Successfully obtained UbPa was confirmed by ESI-MS. UbPa was used to treat HEK293T cell lysates for 12 h at 4 °C first. UbPa-treated HEK293T cell lysates were then reacted separately with FlagUbPa and three ubiquitin azapeptide ester probes for an additional 12 h. All samples were then analyzed by SDS-PAGE and blotted using anti-FLAG (FIG. 6, Panel B). The sample pretreated with UbPa, followed by FlagUbPa, showed only a faint band near 37 kDa, indicating that almost all DUBs capable of reacting with a propargylamine-conjugated ubiquitin probe were inactivated by UbPa. In contrast, the three UbPa-treated samples that were further incubated with ubiquitin azapeptide ester probes displayed distinct bands near 37 kDa (band 1) and 50 kDa (band 2). Additionally, in the FlagUbTFEGHC sample, a unique band around 130 kDa (band 3) was detected. Several other weak bands were also visible in the azapeptide ester-treated samples. The strong intensity of band 1 and the presence of additional bands in the azapeptide ester-treated samples demonstrates that UbPa has low reactivity toward certain enzymes. The successful covalent labeling of these enzymes by the ubiquitin azapeptide ester probes further demonstrates that Ub azapeptide ester probes display superior reactivity toward certain cysteine enzymes compared to FlagUbPa. Consequently, their application in activity-based protein profiling may help identify enzymes that do not react with FlagUbPa.

### Example 5: Proteomic Characterization of Cysteine Enzymes Uniquely Profiled by Ubiquitin Azapeptide Esters

To characterize cysteine enzymes uniquely labeled by the three ubiquitin azapeptide ester probes in UbPa-pretreated HEK293T cell lysates, probe-conjugated proteins were immunoprecipitated using anti-FLAG magnetic agarose beads. The immunoprecipitated proteins were subjected to trypsin digestion, followed by liquid chromatography-tandem mass spectrometry (LC-MS/MS) analysis for peptide sequencing and protein identification. The identified proteins include five deubiquitinating enzymes (DUBs): UCHL1, UCHL3, UCHL5, OTUB1, and OTUD6B. Interestingly, one E2 enzyme, UbE2D3, was also identified and labeled by all three azapeptide ester probes, confirming that ubiquitin azapeptide ester probes can also profile non-DUB cysteine enzymes in the ubiquitination/deubiquitination cascade. A cross-interaction network and a Venn diagram depicting the relationship of these enzymes and their labeling by the three probes are shown in FIG. 6, Panel C and FIG. 6, panel D, respectively. To validate the identification of all six enzymes, SDS-PAGE analysis of anti-FLAG immunoprecipitated samples was performed, followed by western blotting using antibodies specific to UCHL1, UCHL3, UCHL5, OTUB1, OTUD6B, and UbE2D3. As shown in FIG. 6, Panels E-J, clear enrichment of bands corresponding to complexes formed between these enzymes and ubiquitin azapeptide ester probes was observed. Similar bands were absent in the HEK293T cell lysate control sample, underscoring the specificity and efficiency of the azapeptide ester probes in profiling distinct cysteine enzymes involved in the ubiquitin signaling cascade.

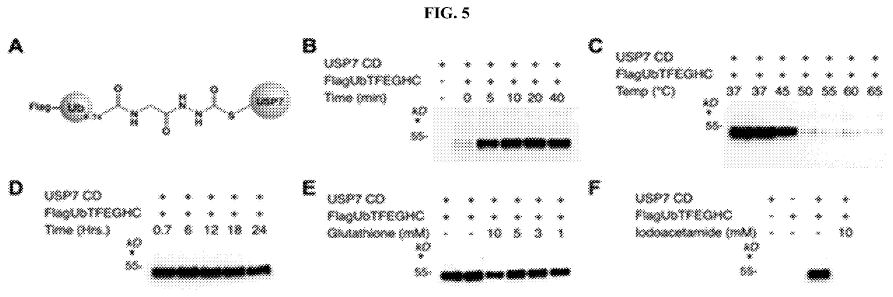

Three DUBs including Ataxin-3, OTULIN, and OTUD7C that exhibited low reactivity toward FlagUbPa but were efficiently labeled by ubiquitin azapeptide ester probes in the biochemical assays shown in FIG. 3 were not identified in the proteomic analysis. This discrepancy may be attributed to their low abundance in HEK293T cells or limitations inherent in the proteomic analysis setup. To directly assess Ataxin-3 labeling by all four probes in HEK293T cell lysates, the lysates were incubated with each probe separately for 20 h, followed by SDS-PAGE and Western blotting using an anti-Ataxin-3 antibody. As shown in FIG. 7, FlagUbPa failed to label Ataxin-3, whereas all three ubiquitin azapeptide ester probes successfully formed covalent complexes with Ataxin-3, highlighting enhanced reactivity and target engagement of azapeptide ester probes compared to FlagUbPa for Ataxin-3 in a cellular environment. Ataxin-3 is a highly specialized DUB that preferentially cleaves a polyubiquitin chain with more than 4 ubiquitin monomers. It also shows high isopeptide linkage selectivity during its catalyzed reactions. The ubiquitin azapeptide ester probes' increased reactivity toward ataxin-3 compared to FlagUbPa is likely due to their better chemical reactivity and stronger binding to the enzyme.

### Example 6: Kinetic Characterizations of Five Identified DUB Enzymes on their Reactions with FlagUbPa and Three Ubiquitin Azapeptides

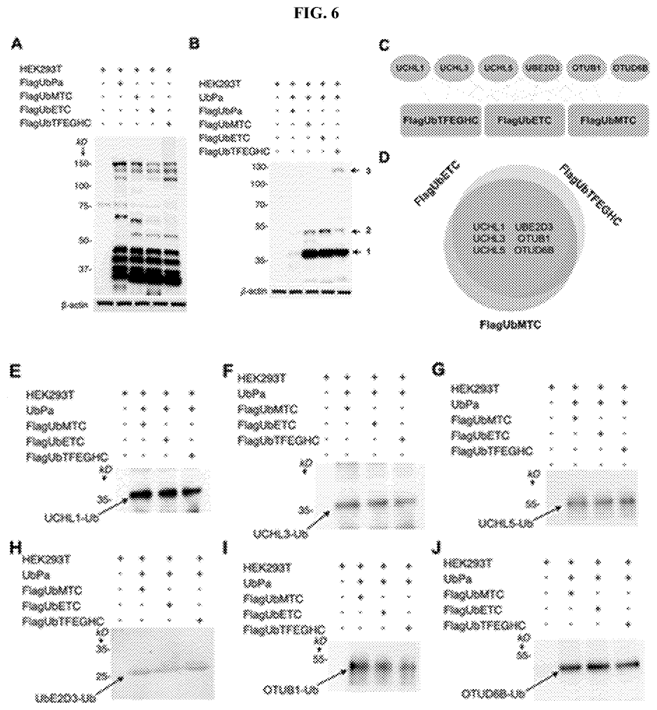

Five DUB enzymes identified in the proteomic analysis, including UCHL1, UCHL5, UCHL3, OTUB1, and OTUD6B, were used to conduct kinetic characterizations of their reactions with FlagUbPa and three ubiquitin azapeptide ester probes. The assay involved incubating a DUB enzyme with a probe for a total reaction time of 40 min at 37 °C and quenching the reaction by adding SDS-PAGE loading dye at various reaction time intervals including 0*(operation dead time<30 s), 5, 10, 20, and 40 min. The quenched reaction samples were then analyzed by SDS-PAGE and blotted using anti-FLAG. Results are shown in FIG. 8. BSA was used as a loading control for this experiment. Compared to FlagUbPa that showed weak labeling with UCHL1 and had close to nondetectable labeling at and before 10 min, all ubiquitin azapeptide ester probes reacted with UCHL1 strongly and displayed clearly visible labeling of UCHL1 at 0*min at which time a DUB enzyme was quickly mixed with a probe and the SDS-PAGE loading dye was added immediately after that (FIG. 8, Panel A). Saturation labeling was observed starting from 5 min for all three ubiquitin azapeptide esters. For FlagUbTFEGHC, strong labeling was detected at 0*min indicating a very rapid reaction between UCHL1 and FlagUbTFEGHC. In comparison, even at 40 min, FlagUbPa displayed labeling of UCHL1 that is clearly not at the saturation level. Like UCHL1, UCHL5 reacted slowly with FlagUbPa and showed weak labeling even at 40 min (FIG. 8, Panel B). However, it displayed labeling with all three ubiquitin azapeptide esters even at 0*min. Starting from 5 min, UCHL5 labeling with FlagUbMTC and FlagUbETC reached saturation. Surprisingly, UCHL5 labeling with FlagUbTFEGHC reached saturation at 0*min, indicating superior reactivity. For UCHL3, its labeling with all four probes reached saturation at 0*min (FIG. 8, Panel C). For OTUB1, its labeling with FlagUbMTC outperformed the other three probes (FIG. 8, Panel D). It exhibited strong labeling with FlagUbMTC at 0*min and the labeling reached saturation at 5 min. For the other three probes, their labeling of OTUB1 continued to rise from 0 to 40 min. For OTUD6B, three ubiquitin azapeptide esters outperformed FlagUbPa in its labeling, although for all probes the labeling continued to rise from 0 to 40 min (FIG. 8, Panel E). Overall, the kinetic labeling results align with the proteomic findings, though it is important to note that binding partners in cell lysates may have significant impacts on the proteomic results.

### Example 7: Inhibitory Effects of FlagUbPa and Ubiquitin Azapeptide Ester Probes on the Enzymatic Activities of UCHL1, UCHL5, and USP25

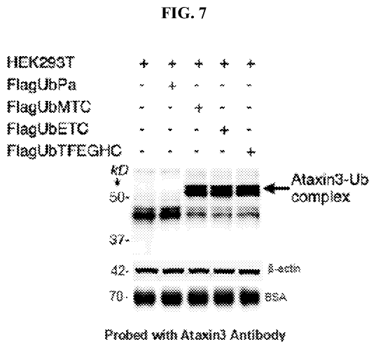

FIG. 8 demonstrates that ubiquitin azapeptide ester probes exhibit significantly stronger labeling of UCHL1 and UCHL5 compared to FlagUbPa. To further investigate this observation, ubiquitin-7-amino-4-coumarinyl-acetic acid (UbACA), shown in FIG. 9, Panel A, was synthesized as a fluorogenic substrate for DUBs. The ACPL reaction was carried out between Ub₁₋₇₅-Cys-6×His and Gly-ACA to make UbACA. Gly-ACA was chosen over Gly-AMC (7-amino-4-methylcoumarin) due to its superior water solubility. Additionally, Ub-AMC is highly expensive to produce, even at microgram scales, making it impractical for routine research use. The successful synthesis of UbACA was confirmed by ESI-MS, which showed a molecular weight consistent with its theoretical value. UbACA serves as a fluorogenic substrate for DUBs, where its catalytic hydrolysis by a DUB releases ACA, a highly fluorescent compound that can be detected using a plate reader. To validate UbACA as a substrate for UCHL1 and UCHL5, 2 μM UbACA was reacted with 20 nM UCHL1 or UCHL5 and time-dependent fluorescence changes were measured (Excitation: 360 nm, Emission: 460 nm). The results, shown in FIG. 9, Panel B, confirm that both UCHL1 and UCHL5 are active toward UbACA. USP25 was also included in this analysis as a control, given its unequal labeling efficiency with the four synthesized FLAG-containing ubiquitin probes as shown in FIG. 3, Panel A (vii). As shown in FIG. 9, Panel B, USP25 is also active toward UbACA. Using UbACA as a substrate, the inhibitory effects of FlagUbPa and three ubiquitin azapeptide ester probes on UCHL1, UCHL5, and USP25 were analyzed. In these assays, 20 nM of each enzyme catalyzed ACA release from 2 μM UbACA in the presence of varying concentrations of one of the four synthetic probes. The initial ACA release velocities were plotted against probe concentrations, with results presented in FIG. 9, Panels C-E. For both UCHL1 and UCHL5, FlagUbPa exhibited very weak inhibition, even at 2 μM, whereas FlagUbTFEGHC achieved nearly 100% inhibition at 300 nM. Both FlagUbMTC and FlagUbETC demonstrated strong inhibition of UCHL1, with each achieving 50% inhibition at 100 nM. Although these two probes showed weaker inhibition of UCHL5 compared to UCHL1, they still achieved approximately 50% inhibition of UCHL5 at 2 μM. For USP25, FlagUbMTC exhibited the highest inhibition potency, followed by FlagUbTFEGHC, FlagUbETC, and finally FlagUbPa, although the differences in inhibition potency among the four probes were less pronounced compared to UCHL1 and UCHL5.

While these kinetic assays were conducted under competitive inhibition conditions, Ki values for each probe were not directly determined, as all four probes covalently react with the enzymes. Nonetheless, the results strongly demonstrate that the ubiquitin azapeptide ester probes act as better mimics of native ubiquitinated protein substrates and exhibit superior interaction with UCHL1, UCHL5, and USP25 compared to FlagUbPa. Additionally, these findings are consistent with the proteomic results.

### Example 8: Activity-Based Labeling of Cysteine Enzymes in the Ubiquitination/Deubiquitination Cascade from Mouse Tissues Using Ubiquitin Azapeptide Ester Probes

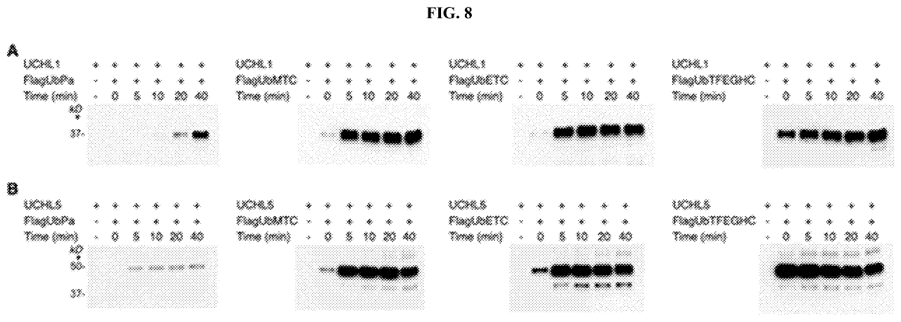

Three ubiquitin azapeptide esters were tested as activity-based probes to label cysteine enzymes in the ubiquitination/deubiquitination cascade from mouse tissues. FlagUbPa was used as a control. Mouse tissues including kidney, brain, liver, and lung were dissected and immediately stored on dry ice. Tissue samples of 20-50 mg were homogenized, clarified by centrifugation, and the resulting supernatants were used to react separately with all four probes for 20 h at 4 °C. Final reaction mixtures were then separated by SDS-PAGE and blotted by anti-FLAG. Results are presented in FIG. 10, Panels A-D. To probe cysteine enzymes that may be uniquely modified by ubiquitin azapeptide esters but not by FlagUbPa, additional labeling experiments were conducted where all mouse tissue lysates were incubated with UbPa for 12 h before a ubiquitin azapeptide ester was added for an additional incubation period of 12 h. Final reaction samples were then separated by SDS-PAGE and blotted by anti-FLAG. Results from these experiments are presented in FIG. 10, Panels E-H. FIG. 10, Panels A-D revealed distinct cysteine enzyme labeling patterns by all four probes across different tissues, indicating different proteomes of cysteine enzymes in the ubiquitination/deubiquitination cascade are expressed in these tissues. Interestingly, ubiquitin azapeptide esters consistently provided increased labeling of cysteine enzymes compared to FlagUbPa in all mouse tissue lysates. Remarkably, at least one ubiquitin azapeptide ester consistently demonstrated superior labeling efficiency compared to FlagUbPa, demonstrating enhanced substrate engagement and reactivity. These results are attributed to the feature that ubiquitin azapeptide esters are better than other conventional activity-based probes in mimicking ubiquitinated proteins when reacting with cysteine enzymes. It is also notable that kidney and liver exhibited higher levels of labeled cysteine enzymes compared to brain and lung. These different labeling patterns may be attributed to diverse metabolic activities and/or cellular processes inherent to each tissue type. Kidney and liver, known for their involvement in detoxification and complex metabolic pathways, require a multitude of complex enzymes. The increased abundance of cysteine enzymes in the ubiquitination/deubiquitination cascade in the two tissues likely contributes to enhanced labeling with all four probes. In contrast, lower levels of labeling observed for brain and lung demonstrate a reduced abundance of cysteine enzymes in the ubiquitination/deubiquitination cascade involved in metabolic and/or other cellular processes. FIG. 10, Panels E-H reveal cysteine proteases that cannot be fully removed by UbPa. All four tissue lysates display cysteine enzymes intensely labeled with ubiquitin azapeptide esters after their treatment with UbPa. There is also a significant number of intensely labeled bands for kidney, lung, and liver. Although brain tissue lysates showed fewer labeled bands, there are still several significantly labeled bands including the one around 37 kDa. Once again, labeling patterns in FIG. 10, Panels E-H are distinct from each other indicating unique cysteine proteomes in four tissues. Comparatively, the results demonstrate that ubiquitin azapeptide esters serve as better activity-based probes than FlagUbPa to pull out more enzymes. However, due to different reactivities of all probes, a combination of ubiquitin probes with different reactive C-terminal groups may be used for even more thorough activity-based protein profiling of cysteine enzymes in the ubiquitination/deubiquitination cascade.

### Example 9: Synthesis of Eleven UBL-ACA Fluorogenic Substrates Using the ACPL Technique

To extend the utility of the ACPL technique beyond ubiquitin, eleven FLAG-tagged UBL-ACA fluorogenic substrates were synthesized. Eleven FLAG-UBL-GxC-6×His expression constructs, where x denotes the terminal glycine position, were generated for ubiquitin-like proteins, including SUMO1, SUMO2, SUMO3, SUMO4, URM1, UFM1, NEDD8, ISG15, GABARAP, GABARAPL2, and MNSFβ. Since ISG15, SUMO1, SUMO2, SUMO3, SUMO4, and MNSFβ natively contain a cysteine residue that may undergo the ACPL reaction, this cysteine was mutated to alanine in all six proteins to ensure site-specific conjugation at the C-terminal cysteine. All eleven FLAG-UBL-GxC-6×His proteins were recombinantly expressed in E. coli BL21(DE3) cells and purified to at least 90% purity using Ni²⁺-NTA affinity chromatography. All purified proteins were analyzed by SDS-PAGE and subjected to electrospray ionization mass spectrometry (ESI-MS) analysis. Deconvoluted ESI-MS spectra confirmed their intact chemical compositions with molecular weights matching theoretical values.

All purified FLAG-UBL-GxC-6×His proteins were then subjected to the ACPL reaction with Gly-ACA. In this one-pot reaction, 500 μM FLAG-UBL-GxC-6×His was mixed with 1 mM TCEP, 5 mM NTCB, and 500 mM Gly-ACA in 1×PBS buffer and incubated overnight at 37 °C. Gly-ACA was well soluble in these reaction conditions, allowing a 500 mM final concentration to be used, in contrast to Gly-AMC which could only achieve a final concentration of approximately 40 mM. A similar reaction was performed to synthesize Ub-ACA following the protocol described in Example 13. All products were purified using FPLC and unreacted proteins were then removed by incubating with Ni-NTA resins. The purified probes were analyzed by SDS-PAGE and subjected to ESI-MS. For all twelve Ub/UBL-ACA products, deconvoluted ESI-MS spectra confirmed successful synthesis, with all products having determined molecular weights that match closely with their theoretical values with a deviation of 0.3 Da. Using Ub-ACA as an example, the product yield was determined as 26%. To confirm that the ACA conjugation does not disrupt protein folding, circular dichroism (CD) spectroscopy was performed on Ub-ACA, which closely resembled the spectrum of native ubiquitin, indicating that the overall protein fold is preserved following ACA conjugation.

The ACPL strategy enabled one-pot, rapid, and efficient generation of all Ub/UBL-ACAs without the need for intein, enzymatic conjugation, or refolding steps, offering a straightforward approach to obtain fluorogenic ubiquitin and ubiquitin-like protein substrates in a time- and labor-efficient manner. This method significantly streamlines the preparation and purification process compared to traditional Ub-AMC synthesis using expressed protein ligation. The synthesis of eleven UBL-ACAs represents the first reported fluorogenic substrates for five of the UBLs tested, namely URM1-ACA, MNSFβ-ACA, GABARAP-ACA, GABARAPL2-ACA, and SUMO4-ACA.

### Example 10: Enzymatic Validation of Ubiquitin-Like ACA Enzymatic Fluorogenic Substrates Using Cognate DUBs and ULPs

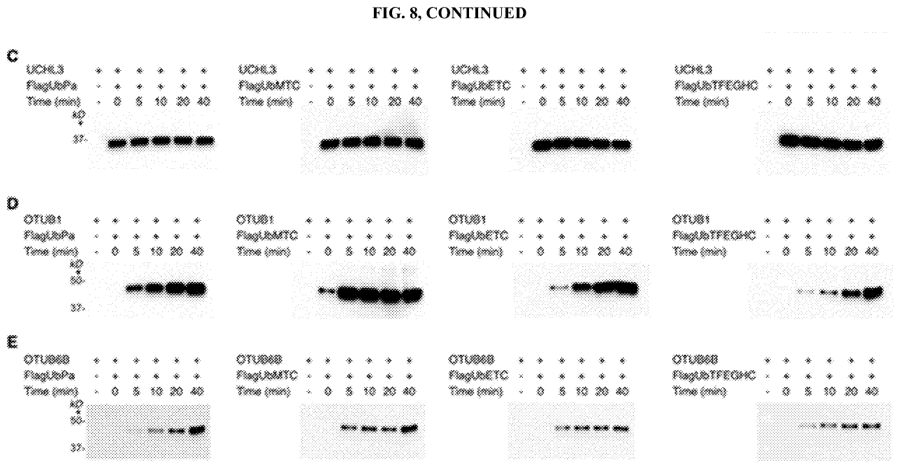

With the successful synthesis of twelve Ub/UBL-ACAs, their utility as fluorogenic substrates for deubiquitinases (DUBs) and ULPs was systematically evaluated using panels of recombinant enzymes. For Ub-ACA, cleavage was tested with a diverse panel of recombinant cysteine DUBs representing different mechanistic subclasses, including UCHL1, UCHL3, UCHL5, USP2, USP5, USP7, USP9X, USP15, and USP21. Each DUB (50 nM) was incubated with 400 nM Ub-ACA at pH 7.6 in assay buffer (50 mM Tris, 100 mM NaCl, 0.5 mM EDTA, 1 mM DTT, 0.1% BSA), and enzymatic cleavage was monitored in real-time by measuring the release of the fluorescent ACA moiety (Excitation: 360 nm; Emission: 460 nm). All tested enzymes belonging to different DUB classes exhibited robust activities to catalyze the release of fluorescent ACA from the fluorogenic Ub-ACA substrate, demonstrating Ub-ACA as a broadly applied substrate for DUBs (FIG. 11).

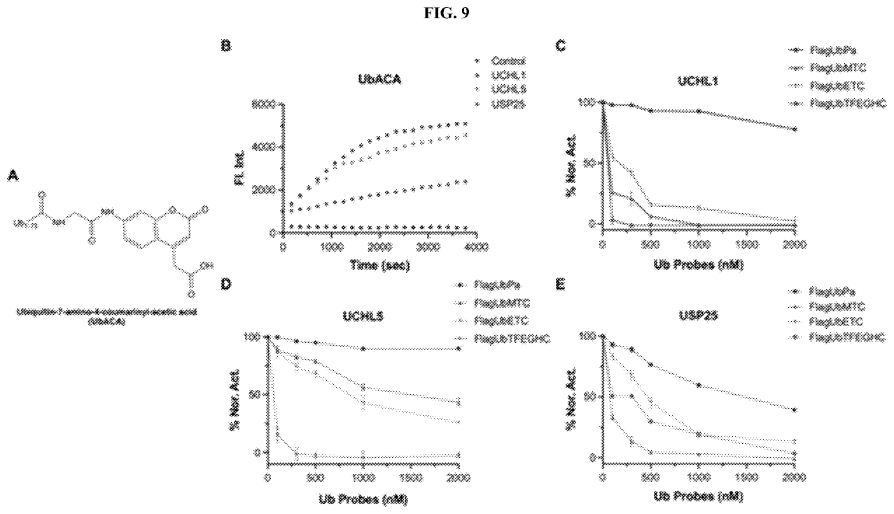

The SENP family enzymes are established SUMO proteases. SENP1 was acquired and tested for its activity on the four synthesized SUMO-ACA substrates. In the fluorescence activity assay, 100 pM SENP1 was incubated with 400 nM of each FLAG-SUMO-ACA substrate in assay buffer (50 mM Tris, 100 mM NaCl, 0.5 mM EDTA, 1 mM DTT, 0.1% BSA, pH 7.6). Enzymatic cleavage was monitored in real-time by measuring the release of the fluorescent ACA moiety (Excitation: 360 nm; Emission: 460 nm). SENP1 displayed robust activities toward all four fluorogenic SUMO-ACA probes, with reactions for all four substrates finishing within 500 seconds (FIG. 12).

Kinetic results indicate that while SENP1's activity toward SUMO4 may be slightly higher than for the other three isoforms, all four SUMO-ACAs are cleaved with essentially comparable efficiencies. This finding is particularly significant because, compared to SUMO1-3, SUMO4 is a less abundant post-translational modification and has more structural differences (particularly in its N-terminal extension) compared to SUMO1-3, which share high sequence identity. The comparable catalytic efficiency of SENP1 toward SUMO4-ACA demonstrates that SENP1 involves recognition of commonly conserved regions in all four SUMO isoforms for almost equally high catalytic activities.

To validate the substrate specificity of the SUMO-ACA probes, potential cross-reactivity between noncognate enzyme-substrate pairs was evaluated. SENP1 was incubated with Ub-ACA, while UCHL1, a deubiquitinase, was tested with SUMO4-ACA. In both cases, no fluorescence signal was detected, indicating the absence of enzymatic cleavage. These results confirm that SENP1 does not process Ub-ACA and UCHL1 does not act on SUMO4-ACA, underscoring the substrate selectivity of the respective enzymes and supporting the specificity of SUMO4-ACA as a probe for SUMO proteases.

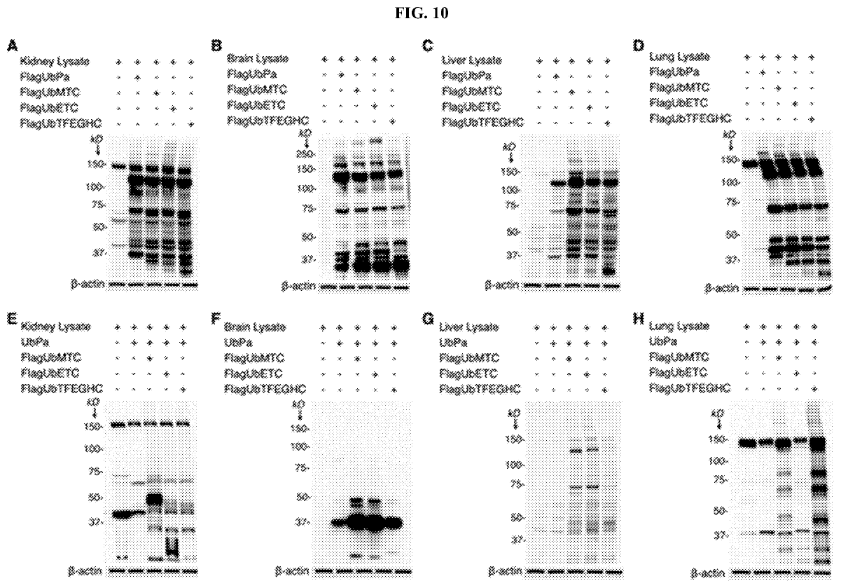

SENP8 and UCHL3 are reported as ULPs of NEDD8. Both of these enzymes, along with USP21, were tested at 50 nM concentration with 400 nM FLAG-NEDD8-ACA. Both SENP8 and UCHL3 showed a time-dependent increase in the fluorescence upon cleavage of FLAG-NEDD8-ACA (FIG. 13). Additionally, USP21 also showed robust enzymatic activity toward the probe (FIG. 13). This observation reflects these enzymes' capability to process NEDD8-conjugated substrates and reveals a dual substrate specificity for UCHL3 and USP21, demonstrating that these enzymes possess the ability to accommodate structurally similar ubiquitin and NEDD8 moieties within their catalytic clefts. To evaluate the substrate specificity of NEDD8-ACA, its reactivity with USP5, a deubiquitinase known to lack NEDD8 cross-reactivity, was tested. USP5 did not exhibit any detectable cleavage of NEDD8-ACA, thereby validating the specificity of the probe and underscoring its suitability as a selective substrate for NEDD8-specific proteases.

ATG4B is a cysteine protease that plays a critical role in the autophagy pathway. ATG4B is known to process autophagy-related UBLs, including LC3 and GABARAP proteins. ATG4B (20 nM) was tested for its activity against FLAG-GABARAP-ACA and FLAG-GABARAPL2-ACA at 400 nM in assay buffer (50 mM Tris, 100 mM NaCl, 0.5 mM EDTA, 1 mM DTT, 0.1% BSA, pH 7.6). Enzymatic cleavage was monitored in real-time by measuring the release of the fluorescent ACA moiety (Excitation: 360 nm; Emission: 460 nm).

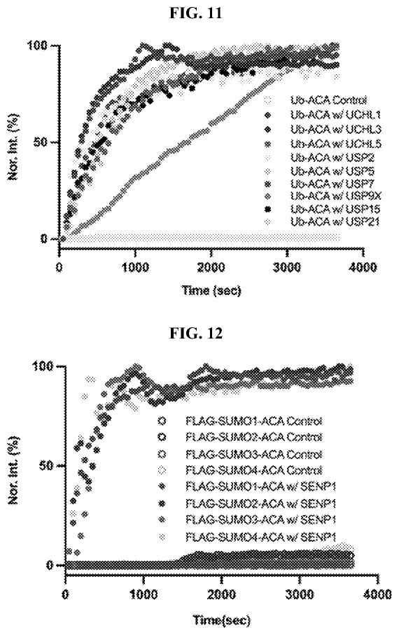

Both probes were efficiently cleaved by ATG4B (FIG. 14). The kinetics exhibited a very interesting trajectory that clearly showed a two-phase process, with the first as a binding phase and the second as a catalytic phase. A substrate-binding triggered allosteric change for substrate-induced activation has been previously observed with ATG4B during crystallographic analysis. The present results are the first to show that this allosteric change can be kinetically traced in real-time using a fluorogenic substrate. The ability of GABARAP-ACA and GABARAPL2-ACA to resolve this two-phase kinetic behavior demonstrates a unique application of these new fluorogenic probes in enzyme mechanistic analysis for DUBs and ULPs.

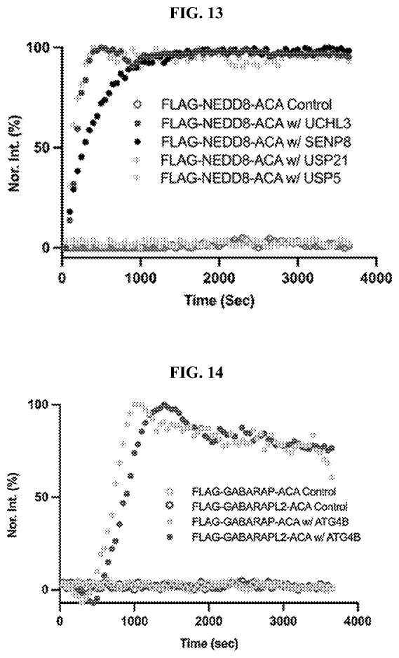

USP18 is reported as a ULP for ISG15. The activity of USP18 was assessed using FLAG-ISG15-ACA at 50 nM enzyme and 400 nM substrate. Cleavage of FLAG-ISG15-ACA by USP18 resulted in a strong fluorescence emission, confirming USP18's specificity toward ISG15-conjugated substrates (FIG. 15).

UFSP2 is known as a ULP for UFM1. FLAG-UFM1-ACA was employed to assess UFSP2's catalytic activity at 50 nM enzyme and 400 nM substrate. Upon cleavage by UFSP2, the release of ACA resulted in increased fluorescence (FIG. 15). This reaction confirms UFSP2's role in processing UFM1 precursors and its specificity for UFM1 over other ubiquitin-like modifiers.

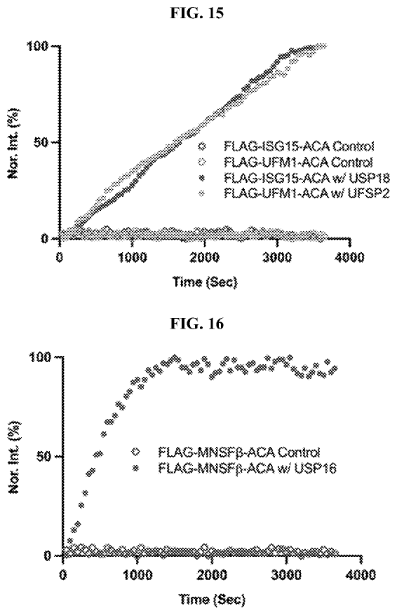

USP16 has been reported as a ULP for MNSFβ. Its activity was analyzed using FLAG-MNSFβ-ACA at 50 nM enzyme and 400 nM substrate. Incubating USP16 with FLAG-MNSFβ-ACA led to robust ACA cleavage, supporting that posttranslational MNSFβ modification is likely regulated by USP16 (FIG. 16).

### Example 11: Profiling of DUB and ULP Activities in HEK293T Cell Lysates Using all Twelve Ub/UBL-ACA Probes

To evaluate the use of all twelve Ub/UBL-ACAs as probes for DUBs and ULPs in a biologically relevant cellular environment, HEK293T cells were chosen as a model human cell line. HEK293T cells were cultured, collected, and lysed following established protocols. Cell lysates with an overall protein load of 100 μg were incubated with all twelve Ub/UBL-ACAs at 400 nM concentration separately at 30 °C. Fluorescent release of ACA was monitored continuously for 90 minutes.

All twelve Ub/UBL-ACAs were processed by HEK293T cell lysates, indicating that DUBs or ULPs exist for ubiquitin and all eleven UBLs tested. Differential processing rates were observed across the probe panel. Ub-ACA and SUMO1-4-ACAs were completely hydrolyzed within 1 hour, consistent with the high abundance and well-studied roles of the ubiquitin and SUMO pathways. Other UBL-ACAs including URM1-ACA, UFM1-ACA, NEDD8-ACA, ISG15-ACA, GABARAP-ACA, GABARAPL2-ACA, and MNSFβ-ACA were processed much more slowly, reflecting their lower abundance in cells.

To confirm that hydrolysis of Ub-ACA is mainly from cysteine DUBs, two further control experiments were performed. HEK293T cell lysates were pretreated with N-ethylmaleimide (NEM) to covalently block cysteine residues in cysteine enzymes, or with EDTA to sequester metal ions from metalloenzymes. These two pretreated cell lysates were then incubated with Ub-ACA to monitor ACA release. NEM treatment completely inhibited Ub-ACA hydrolysis, indicating that cysteine DUBs are the major ubiquitin proteases in cells. EDTA-treated cell lysates showed a similar Ub-ACA hydrolysis trend as untreated lysates, demonstrating that metalloprotease-mediated hydrolysis contributes minimally to the observed activity. The combined results with NEM and EDTA controls confirm that cysteine-dependent DUBs are the primary enzymes responsible for Ub-ACA processing in HEK293T cell lysates.

Cell lysate-catalyzed hydrolysis of four SUMO-ACAs presented a very similar trend. In combination with results from SENP1-catalyzed hydrolysis of four SUMO-ACAs shown above, these results further demonstrate that SUMO1-4 share similar regulatory mechanisms within the cellular environment.

### Example 12: Discovery of URM1-Specific Protease Activity in Human Cell Lysates

URM1 is distinct from ubiquitin and other UBLs in that it does not have a conventional E1-E2-E3 cascade. URM1 is activated by an E1-like enzyme in both yeast and human cells to form a C-terminal thiocarboxylate that serves two roles: one to transfer sulfur for tRNA thiolation and the other for direct conjugation with substrate protein lysines. Critically, there are no known ULPs reported for URM1 in either yeast or human cells.

Enzymatic cleavage of FLAG-URM1-ACA was observed in HEK293T cell lysates. URM1-ACA was processed by HEK293T cell lysates with slower kinetics compared to Ub-ACA and SUMO-ACAs. This reflects the expected lower abundance of URM1-processing enzymes. These findings demonstrate the presence of URM1-processing proteases, herein termed deurmylase(s), in human cells. However, the identities of these proteases remain to be elucidated.

To determine whether the enzymatic activity observed toward URM1-ACA in human cell lysates arises from cysteine-based enzymes, an N-Ethylmaleimide (NEM) quenching assay was performed. Pretreatment of HEK293T cell lysates with 1 mM NEM, a broad-spectrum cysteine protease inhibitor, completely abolished URM1-ACA cleavage activity. This result demonstrates that the enzymatic processing of URM1-ACA is mediated by a cysteine-dependent protease.

Since the identity of the putative deurmylase(s) remains unknown, the use of crude cell lysates was essential to capture endogenous activity and demonstrate that URM1-processing enzymes are indeed present in human cells. This cell-based strategy provides a functional readout in the absence of a defined recombinant enzyme, reinforcing the utility of URM1-ACA as a substrate to profile and identify cysteine-based deurmylases in complex biological systems. The discovery of URM1-specific protease activity represents the first experimental evidence that deurmylase(s) exist in human cells and establishes URM1-ACA as a critical tool for their identification and characterization.

### Example 13: Materials and Methods

Compounds: Methyl hydrazinecarboxylate (MTC) and Ethyl hydrazinecarboxylate (ETC), TCEP and NTCB were purchased from A2B chem LLC, San Diego, CA, USA. Propargylamine was purchased from TCI Chemicals, America.

Expression and Purification of All Ubiquitin Proteins: All ubiquitin proteins including Ub₁₋₇₅-Cys-6×His, Flag-Ub₁₋₇₄-Cys-6×His, and Flag-Ub₁₋₇₅-Cys-6×His were recombinantly expressed and purified. An overnight culture of E. coli BL21(DE3) cells harboring a pETDuet-1 expression vector coding Ub₁₋₇₅-Cys-6×His, Flag-Ub₁₋₇₄-Cys-6×His, or Flag-Ub₁₋₇₅-Cys-6×His was inoculated (1:100 dilution) into a 2×YT medium containing 100 μg/mL ampicillin. Cells were grown at 37 °C until OD₆₀₀ reached 0.6-0.9 and 1 mM IPTG was added to induce protein expression. The cells were grown in a shaker at 37 °C for 8 h or 18 °C for 16 h and collected by centrifugation (5,000 rpm, 30 min, 4 °C). The cell pellets were resuspended in a 100 mL lysis buffer (50 mM NaH₂PO₄, 500 mM NaCl, 5 mM imidazole, 1 mM TCEP, pH 7.8) and lysed by sonication. The cell lysates were clarified by centrifugation (10,000 rpm, 30 min, 4 °C) and the supernatant was decanted. 6 M HCl solution was then added dropwise into the supernatant to adjust pH to 2 to drive the formation of white precipitate inside the solution. The precipitate was pelleted by centrifugation (10,000 rpm, 30 min, 4 °C) and removed. The pH value of the supernatant was then adjusted back to 7.8 by adding 6 M NaOH. This supernatant was loaded onto high-affinity Ni²⁺-NTA resins at RT and then washed with a buffer (50 mM NaH₂PO₄, 500 mM NaCl, 30 mM imidazole, 1 mM TCEP, pH 7.8). The bound protein was eluted by another buffer (50 mM NaH₂PO₄, 250 mM NaCl, 300 mM imidazole, 1 mM TCEP, pH 7.8) and then desalted into a 50 mM ammonium bicarbonate (ABC) buffer using HiPrep 26/10 Desalting column (Cytiva). All proteins were concentrated using 3 kDa MWCO Amicon ultracentrifugation filter devices (Millipore Sigma) and concentrations were measured using a NanoDrop spectrophotometer (ThermoFisher). Eventually, all proteins were aliquoted, lyophilized, and stored at −80 °C.

Use of ACPL to Synthesize Ubiquitin Azapeptide Ester Probes: A 500 mM TCEP stock solution was prepared in water, and a 500 mM NTCB stock solution in DMSO. Solutions of Propargylamine (Pa), Methyl hydrazinecarboxylate (MTC), and Ethyl hydrazinecarboxylate (ETC) were made in water at 2 M each, while a 2 M solution of 2,2,2-trifluoroethyl-2-glycylhydrazine-1-carboxylate (TFEGHC) was prepared in DMSO.

Synthesis of FlagUbMTC and FlagUbETC. To initiate the reaction, Flag-Ub₁₋₇₅-Cys-6×His protein pellets were dissolved in MTC and ETC solutions. TCEP and NTCB were added to a final concentration of 5 mM each, and the pH was adjusted to 8. The mixtures were incubated at 37 °C, desalted using a HiTrap column, and then incubated with Ni²⁺-NTA resins and were removed with micro syringe filters, and the flowthroughs were collected for ESI-MS analysis.

Synthesis of FlagUbTFEGHC. Flag-Ub₁₋₇₄-Cys-6×His protein pellets were dissolved in water, followed by the addition of TFEGHC, TCEP (5 mM), and NTCB (5 mM). The pH was adjusted to 8 and the mixture was incubated at 37 °C. The reaction was desalted using a HiTrap column, then incubated with Ni²⁺-NTA resins and were removed with micro syringe filters, and the flowthroughs were collected for ESI-MS analysis.

Synthesis of FlagUbPa and UbPa. Flag-Ub₁₋₇₅-Cys-6×His and Ub₁₋₇₅-Cys-6×His protein pellets were dissolved in 500 mM propargylamine, with TCEP and NTCB added (5 mM each). The pH was adjusted to 9 and the mixtures were incubated at 37 °C. After desalting with a HiTrap column, the products were incubated with Ni²⁺-NTA resins and were removed with micro syringe filters, and the flowthroughs were collected for ESI-MS analysis.

Western Blot Analysis for FlagUbPa and Ubiquitin Azapeptide Ester Probes: To assess the functionality of the Flag tag linked to the synthesized Ubiquitin probes, all probes underwent SDS-PAGE (mPAGE 4-12% bis-tris gel, Millipore Sigma) followed by western blotting. A peroxidase (HRP)-conjugated anti-FLAG antibody (Millipore Sigma), capable of binding the Flag tag, was utilized for detection.

Covalent Capture of USPS by Ubiquitin Azapeptide Ester Probes: 0.1 μg/L USP5 (Sino Biological) and 0.1 μg/μL all ubiquitin activity-based probes were mixed, and the final reaction volume was adjusted into a 20 μL DUB reaction buffer (50 mM HEPES, 100 mM NaCl, 5 mM TCEP pH 7.85). All reactions were incubated at 37 °C for 40 min and quenched by adding a 6×SDS loading dye containing 30% β-mercaptoethanol. The reaction mixtures were then analyzed by SDS-PAGE (4-12% bis-tris gel) and Western blotting using HRP-conjugated anti-FLAG.

Covalent Complex Formation between FlagUbPa, Ub Azapeptide Probes, and DUBs: 0.1 μg/μL USP2 CD (R&D systems), USP7 CD (Medchem express), USP8 (Boston biochem), USP10 (R&D systems), USP14 (UBP bio), GST-USP15 (UBP bio), USP25 (R&D systems), CYLD (Sino biological), UCHL3 (UBP bio), GST-Ataxin3 (UBP bio), GST-JOSD2 (UBP bio), OTUB2 (UBP bio), OTULIN (UBP bio), Cezanne-OTUD7B (UBP bio), OTUD2 (UBP bio), OTUD7C (UBP bio) and 0.1 μg/μL all ubiquitin probes were mixed, and the final reaction volume was adjusted into a 20 μL DUB reaction buffer (50 mM HEPES, 100 mM NaCl, 5 mM TCEP pH 7.85). All reactions were incubated at 37 °C for 40 min and quenched by adding a 6×SDS loading dye containing 30% β-mercaptoethanol. The reaction mixtures were then analyzed by SDS-PAGE (4-12% bis-tris gel) and Western blotting using HRP-conjugated anti-FLAG.

The Reaction of all DUBs with Biotin-GG-LRG-MTC Peptide: 0.1 μg/L of each of the DUBs (mentioned above) and the peptide were mixed, and the final reaction volume was adjusted into a 20 μL DUB reaction buffer (50 mM HEPES, 100 mM NaCl, 5 mM TCEP pH 7.85). All reactions were incubated at 37 °C for 40 min and quenched by adding a 6×SDS loading dye containing 30% β-mercaptoethanol. 2 μM streptavidin was added to each reaction mixture followed by SDS-PAGE (4-12% bis-tris gel) and Western blotting using anti-streptavidin antibody (R&D systems) and detected using an anti-rabbit secondary antibody (Abcam).

Labeling of E1 Activating Enzyme: E1 labeling experiments were performed in a ubiquitination buffer containing 50 mM HEPES, 100 mM NaCl, 5 mM TCEP (pH 7.85), 0.1 μg/μL UBE1 (UBP bio) and ubiquitin probes. The reaction mixtures were incubated at 37 °C. Reactions were quenched by adding a 6×SDS loading dye containing 30% 3-mercaptoethanol. The mixtures were then analyzed by SDS-PAGE (4-12% bis-tris gel) and Western blotting using HRP-conjugated anti-FLAG.

Labeling of E2 Conjugating Enzyme: E2 labeling experiments were performed in a ubiquitination buffer containing 50 mM HEPES, 100 mM NaCl, 5 mM TCEP (pH-7.85), 0.1 μg/μL UBE2E2 (UBP bio), and ubiquitin probes. The reaction mixtures were incubated at 37 °C. Reactions were quenched by adding a 6×SDS loading dye containing 30% β-mercaptoethanol. The mixtures were then analyzed by SDS-PAGE (8-16% bis-tris gel) and Western blotting. The blot was probed with an anti-UBE2E2 primary antibody (Invitrogen) derived from rabbit and detected using an HRP-conjugated goat anti-rabbit secondary antibody (Abcam).

Labeling of Parkin: Parkin was recombinantly expressed in E. coli. Labeling of Parkin was performed in a ubiquitination buffer containing 50 mM HEPES, 100 mM NaCl, 5 mM TCEP (pH—7.85), 0.1 μg/μL Parkin, and ubiquitin probes. The reaction mixtures were incubated at 37 °C. Reactions were quenched by adding a 6×SDS loading dye containing 30% β-mercaptoethanol. The mixtures were then analyzed by SDS-PAGE (4-12% bis-tris gel) and Western blotting using HRP-conjugated anti-FLAG.

In vitro Labeling of UBE3A and ITCH. Labeling experiment of UBE3A was performed in a ubiquitination buffer containing 50 mM HEPES, 100 mM NaCl, 5 mM TCEP (pH 7.85), 0.1 μg/μL UBE3A (R&D Systems), ITCH (UBP bio), and the ubiquitin probes. The reaction mixtures were incubated at 37 °C before they were quenched by adding a 6×SDS loading dye containing 30% β-mercaptoethanol. The mixtures were then analyzed by SDS-PAGE (4-12% bis-tris gel) and Western blotting using HRP-conjugated anti-FLAG.

Profiling of Active Ubiquitination/Deubiquitination Cysteine Enzymes in the HEK293T cell lysate using Ubiquitin Probes: HTEK293T cells were cultured in DMEM (Gibco) supplemented with 10% fetal bovine serum (FBS, Gibco) and 5% penicillin-streptomycin (Pen-Strep, Sigma; Pen: 10,000 units/Strep: 10 mg/mL). The cells were grown in T75 flasks in a 37 °C, 5% CO₂ incubator until they reached approximately 90% confluency. Subsequently, the cells were collected in 1×PBS buffer and transferred into 1.5 mL tubes. To lyse the cells, Pierce (ThermoFisher) IP protease-free cell lysis buffer was added to the cell suspension. The mixture was gently shaken under refrigeration for 30 min to ensure cell lysis. After the cell lysis process, the cell lysate was clarified by centrifugation at 14,000 rpm for 30 min at 4 °C. The resulting supernatant was collected, and the protein concentration was measured by the BCA assay.

Profiling Using Four Flag-Fused Ubiquitin Activity-based Probes. HTEK293T cell lysates were incubated separately with one of four probes for 20 h at 4 °C before they were analyzed using a 4-12% bis-tris gel and subjected to Western blotting using HRP-conjugated anti-FLAG.

Profiling After depleting UbPa-binding Cysteine Enzymes. In a separate set of profiling experiments, four aliquoted HEK293T cell lysates were initially incubated with UbPa for 12 h at 4 °C. Afterward, three Flag-fused ubiquitin azapeptide ester probes were added separately to three different reaction mixtures, and the incubation continued for another 12 h at 4 °C. Subsequently, all reactions were analyzed using a 4-12% bis-tris gel followed by Western blotting with HRP-conjugated anti-FLAG.

Immunoprecipitation: Anti-DYKDDDDK magnetic agarose beads (Pierce, Thermo Scientific) were equilibrated to room temperature and thoroughly mixed. A 50 μL slurry of beads was transferred to a microcentrifuge tube and washed twice with binding buffer (25 mM Tris-HCl pH 7.4, 150 mM NaCl, 1 mM EDTA, 1% NP-40, 5% glycerol). Probe-treated cell lysates were added and incubated at room temperature with mixing. The beads were then washed three times with 1×PBS and once with purified water. Bound proteins were acid-eluted, and pH was adjusted with neutralization Buffer. The elution was repeated three times for maximum recovery.

Proteomic Identification of Ubiquitination/Deubiquitination Cysteine Enzymes in HEK293T Cell Lysates: Sample preparation. The eluted proteins from the previous immunoprecipitation procedure were reduced with 25 mM Dithiothreitol at 70 °C for 20 min and alkylated with 50 mM iodoacetamide at room temperature for 30 min in the dark. To acidify samples, 12% phosphoric acid was added to achieve a final concentration of 1.2%. The acidified samples were applied to the S-Trap columns (Protifi, C02-micro) pre-loaded with 165 μl 100 mM TEAB (pH 7.1) in methanol, followed by 5 washes using 150 μl 100 mM TEAB pH 7.1 in methanol. After the last wash, the protein samples underwent on-column digestion with trypsin solution (2 g trypsin in 25 mM TEAB pH 8.0) for 14 hrs at 37 °C. The resulting peptides were subsequently eluted with 40 μl each of 50 mM TEAB (pH 8.0), 0.2% formic acid, and 0.2% formic acid in 50% acetonitrile. The eluted peptides were dried using speed vac and reconstituted in 1% formic acid and 2% acetonitrile, and they were applied to mass spectrometry analysis.

MS data analysis. The peptide samples were analyzed by an Orbitrap Exploris™ 480 mass spectrometer (Thermo Fisher Scientific, Inc., Waltham, MA) coupled with an EASY-nLC 1000 system (Thermo Fisher Scientific, Inc., Waltham, MA). Peptides were separated by a home-made silica column (12 cm length×3 μm ID) packed with C18 resin (Dr. Maisch GmbH, Beim Bruckle, Germany) using solvents A (0.1% formic acid in water) and B (0.1% formic acid in 97% acetonitrile), with a continuous gradient from 2 to 45% B over 90 min, at a flow rate of 200 ng/min. Full-scan mass spectra were surveyed from 300 to 1,100 using the Orbitrap mass analyzer with mass resolution of 60,000 under positive-ion mode. MS/MS was performed in a data-dependent mode, and the spectra were recorded in profile mode. The top 20 most intense ions were selected for MS/MS analysis at a resolution of 15,000 using higher energy collision-induced dissociation (HCD) with 30% normalized collision energy (NCE). Other relevant parameters used in the analysis: isolation window, 1.6 m/z; default charge, 2+; automatic gain control (AGC) target, standard; maximum IT, automatic; dynamic exclusion, automatic.

MS/MS spectra were analyzed by Proteome Discoverer (ver. 2.5, Thermo Fisher Scientific). Mass accuracy was recalibrated by the spectrum files RC node, and the Minora feature detector node was used to detect chromatographic peaks. All MS/MS spectra were searched against a reverse-concatenated human protein sequence database (UniProt-SwissProt, downloaded in March 2021) using SEQUEST HT node. Key criteria used for the SEQUEST HT data searching full trypsin enzyme specificity, maximum missed cleavages of 2, precursor mass tolerance of 10 ppm, fragment mass tolerance of 0.02 Da, static carbamidomethylation of cysteine, and dynamic modifications of methionine oxidation, protein N-terminal Met-loss and Met-loss+acetyl, and lysine acetylation. Spectra matches were evaluated by Percolator with a false discovery rate (FDR) cutoff of <1% based on the reverse sequences of the selected UniProt-SwissProt database.

In the consensus workflow, the feature mapper node was selected for chromatographic retention time alignment between runs with maximum retention time window of 10 min with mass tolerance of 5 ppm. Peptide abundances were normalized using Total Peptide Amount method. A minimum of 1 unique peptide was required for each protein group. Peptides containing dynamic modifications were excluded from protein quantification.

Validation of Cysteine Enzymes Identified in Proteomic Analysis: The eluates from co-immunoprecipitation of UbPa-treated HEK293T cell lysates, followed by Flag-fused ubiquitin azapeptide probes, were analyzed using a 4-12% bis-tris gradient gel and Western blotting. The blot was probed with anti-UCHL1 (Invitrogen), anti-UCHL3 (Proteintech), anti-UCHL5 (Invitrogen), anti-UbE2D3 (Abcam) and anti-OTUD6B antibodies from rabbit, detected with an HRP-conjugated goat anti-rabbit secondary antibody (Abcam). To confirm the presence of OTUB1, anti-OTUB1 (Origene) from mouse was used, detected with an HRP-conjugated anti-mouse secondary antibody (R&D Systems).

Kinetic Characterization of DUBs with Synthesized Ubiquitin Probes: A time dependent reaction between 5 DUBs: OTUB1 (Sino biological), UCHL1 (Sino Biological), UCHL3 (UBP bio), UCHL5 (UBP bio) and OTUD6B (UBP bio) and each of the four Flag-fused ubiquitin probes was carried out. Each reaction mixture comprised 0.1 μg/μL probes and DUBs, and 1 mM DTT in a 50 μL 1×PBS buffer. Additionally, control reactions, lacking the probe but containing the DUB, were prepared. All reactions underwent incubation at 37 °C for various durations: 0, 5, 10, 20, and 40 min. Following each time interval, the reactions were stopped by adding a 6×SDS loading dye containing 30% β-mercaptoethanol. Subsequently, the samples were analyzed using 4-12% bis-tris gel and subjected to Western blotting with HRP-conjugated anti-FLAG.

Synthesis of UbACA using ACPL: Ub₁₋₇₅-Cys-6×His protein pellets were dissolved in 500 mM 2-(7-(2-aminoacetamido)-2-oxo-2H-chromen-4-yl) acetic acid with TCEP and NTCB added (5 mM each). The pH was adjusted to 8.5 and the mixtures were incubated at 37 °C. After desalting with a HiTrap column, the products were incubated with Ni²⁺-NTA resins and were removed with micro syringe filters, and the flowthroughs were collected for ESI-MS analysis. At this stage, hydrolyzed Ub and the probe UbACA were observed. UbACA was further purified by ion-exchange chromatography and the mass was confirmed by ESI-MS analysis.

Characterization of Inhibitory effects of Flag-fused probes on the enzymatic activity of DUBs: In a 96-well plate, 20 nM of each DUB, UCHL1 (Sino Biological), UCHL5 (UBP Bio), and USP25 (R&D Systems) was incubated separately with 2 μM UbACA. Following this, increasing concentrations of Flag-fused Ub probes (100 nM, 300 nM, 500 nM, 1 μM, and 2 μM) were added. Fluorescence measurements were taken at 30 °C for 1 hour (excitation at 360 nm and emission at 460 nm).

Labeling of Ataxin-3 by ubiquitin Probes in HEK293T Cell Lysates: HBEK293T cell lysates were incubated separately with four Flag-fused ubiquitin activity-based probes for 20 h at 4 °C. The samples were then analyzed by SDS-PAGE (4-12% bis-tris gel) and Western blotting. The blot was probed with an anti-ataxin-3 primary antibody (Invitrogen) derived from rabbit and detected using an HRP-conjugated goat anti-rabbit secondary antibody (Abcam).

Mapping Activities of Ubiquitination/Deubiquitination Cysteine Enzymes in Mouse tissue Lysates: Mouse tissues were dissected, followed by immediate placement on dry ice. After weighing the tissue samples, only 20-50 mg per sample was taken for preparing lysates. 20 μL of 1×RIPA buffer was added per milligram of tissue (400-1000 μL total). Tissues were homogenized using a tissue homogenizer. The homogenized tissue lysates were centrifuged at 12,000 RPM at 4 °C for 15 min. The supernatant was decanted and transferred into a clean tube. For the binding reactions, four different mouse tissue lysates (Liver, lungs, kidney, and brain) were incubated separately with each activity-based probe for 20 h at 4 °C. The reaction was stopped by adding a 6×SDS loading dye containing β-mercaptoethanol. Finally, all reactions were analyzed using a 4-12% bis-tris gel and subjected to Western blotting using HRP-conjugated anti-FLAG. In a separate set of binding reactions, four aliquoted mouse tissue lysates were initially incubated with UbPa for 12 h at 4 °C. Afterward, three Flag-fused ubiquitin azapeptide ester probes were added separately to three different reaction mixtures, and the incubation continued for another 12 h at 4 °C. The reaction was terminated by adding a 6×SDS loading dye containing 30% β-mercaptoethanol. Subsequently, all reactions were analyzed using SDS-PAGE (4-12% bis-tris gel) and subjected to Western blotting using HRP-conjugated anti-FLAG.

Biotin-GG-LRG-Methyl Carbazate Synthesis: Glycine preloading to 2-chlorotrityl chloride resin. 2-chlorotrityl chloride resin (AAPPTEC #RTZ001) was swelled with 1.5 mL of degassed dichloromethane (DCM) for 5 min with gentle shaking. Fmoc-glycine (3 eq) and DIPEA (4 eq) were added to the swelled resin slurry and left to react for 5 min. Following the 5 min incubation, an additional 8 eq of DIPEA was added and the reaction was left for 1 h. After the coupling, the residual trityl groups on resin were encapped with the addition of methanol and mixing for 15 min. The resin was drained and washed with DCM (3×3 mL), DMF (3×3 mL), and methanol (3×3 mL). The glycine-loaded resin was then subjected to automated peptide synthesis.

Automated Peptide Synthesis. The remaining amino acids, Gly-Gly-Leu-Arg, were coupled to the glycine-loaded 2-chlorotrityl chloride resin using a MultiPep 2 Peptide Synthesizer (CEM). All amino acid derivatives were standard derivatives for Fmoc peptide synthesis commercially purchased from Chem-Impex. Fmoc-amino acids were deprotected using 20% piperidine in DMF (2×2 mL, 5 min at 50 °C). Amino acids were coupled using HATU/NMM double coupling cycles (4.2 eq amino acid, 4.2 eq HATU, 8 eq NMM, 15 min at 50 °C). Following synthesis, the resin was dried with ethanol washes (3×3 mL) followed by dichloromethane washes (3×3 mL) and evaporated under vacuum.

On-resin Peptide Biotinylation. Following the automated peptide synthesis, the resin was washed with DMF (3×3 mL), and 3 eq of Biotin-NHS (Chem Scene #CS-5714) in 1 mL of DMF was added. After, 6 eq of DIPEA was added to the mixture and the reaction was left overnight with gentle shaking. Biotinylation of the N-terminus was verified with a ninhydrin test.

Peptide Cleavage. The peptide was cleaved in 2 mL of 92.5:2.5.2.5:2.5 TFA:TIS:DODT:H2O for 1 h at room temperature. Following cleavage, the resin was filtered, and the peptide was precipitated from the filtrate by an 8-fold dilution in cold diethyl ether. After collection by centrifugation (5251×g, 20 min), it was lyophilized to a powder overnight.

Methyl Carbazate Coupling. The peptide was dissolved in 1 mL of DMF before the addition of 6 eq methyl carbazate, 1.2 eq HATU, and 3 eq DIPEA. The reaction was left for 1.5 h at room temperature. Following the reaction, the peptide solution was precipitated by an 8-fold dilution in cold diethyl ether. After centrifugation (5251×g, 20 min), the peptide product was lyophilized to a powder overnight. The powder was dissolved in DMF and purified via preparative LC-MS.

### Synthesis of 2,2,2-Trifluoroethyl-2-glycylhydrazine-1-carboxylate

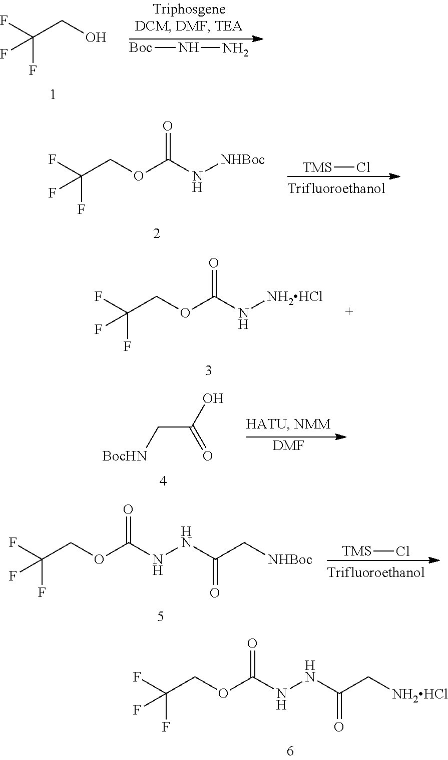

1-(tert-Butyl) 2-(2,2,2-trifluoroethyl) hydrazine-1,2-dicarboxylate (2): To a stirred solution of 2,2,2-trifluoroethanol 1 (7.27 mL, 101.09 mmol), triphosgene (10 g, 33.7 mmol), and dichloromethane (120 mL), Triethyl amine (14.07 mL, 101.09 mmol) was added dropwise at 0 °C for 10 min and stirred for 4 h at RT. Then, the mixture was filtered and washed with 2×20 mL of dichloromethane. The filtrate was then added dropwise into another round bottom flask which was equipped with a magnetic stir bar and charged with tert-butyl hydrazine carboxylate (13.36 g, 101.09 mmol), triethylamine (35.20 mL, 252.67 mmol), and dichloromethane (100 mL) at 0 °C for 1h. Then the reaction mixture was stirred at 0 °C for another 30 min and then 3 h at RT. After completion of reaction, water (100 mL) was added to the reaction mixture, the aqueous and organic phases were separated. The organic layer was washed with 1M HCl (2×50 mL), the organic layer was dried with sodium sulfate, filtered, and concentrated under reduced pressure to get crude residue which was purified by column chromatography (10-20% EtOAc in hexanes as the eluent) to afford 2 as a white solid (8.35 g, 96%). ¹H NMR (400 MHz, DMSO) δ 9.45 (s, 1H), 8.93 (s, 1H), 4.70 (q, J=9.1 Hz, 2H), 1.40 (s, 9H). ¹³C NMR (101 MHz, DMSO) δ 155.45, 155.05, 127.75, 124.99, 122.23, 119.47, 79.50, 60.67, 60.32, 59.97, 59.62, 28.05. ¹⁹F NMR (377 MHz, DMSO) δ −72.85.

2,2,2-Trifluoroethyl hydrazinecarboxylate hydrochloride (3): To a solution of compound 2 (8.3 g, 32.14 mmol) in trifluoroethanol (66.4 mL) was added trimethylsilyl chloride (12.45 mL) slowly at 0 °C, and the resulting solution was stirred for 2 h at RT under an inert condition. After completion of the reaction, as indicated by TLC, solvent was removed under reduced pressure to yield compound 3 as a white solid (6.06 g, 97%). ¹H NMR (400 MHz, DMSO) δ 10.66 (d, J=215.8 Hz, 3H), 4.82 (q, J=9.0 Hz, 2H). ¹³C NMR (101 MHz, DMSO) δ 154.18, 127.52, 124.76, 122.00, 119.24, 61.48, 61.13, 60.78, 60.43. 19F NMR (377 MHz, DMSO) δ −72.80.

2,2,2-Trifluoroethyl 2-((tert-butoxycarbonyl)glycyl)hydrazine-1-carboxylate (5): To a stirred solution of 3 (6.0 g, 30.8 mmol) and 4 (6.47 g, 36.93 mmol) in anhydrous DMF (64 mL) was added NMM (9.35 g, 92.4 mmol) slowly and cooled to 0 °C. HATU (17.56 g 46.2 mmol) was added to the reaction mass at 0 °C and then the reaction mixture was warmed to RT and stirred for 12 h. The reaction mixture was then diluted with ethyl acetate (100 mL) and washed with saturated NaHCO₃ solution (2×50 mL), 1 M HCl solution (2×50 mL), and saturated brine solution (2×50 mL) sequentially. The organic layer was dried over anhydrous Na₂SO₄ and then concentrated in vacuo. The residue was then purified by flash chromatography (50-80% EtOAc in hexanes as the eluent) to afford 5 as white solid (7.76 g, 80%). ¹H NMR (400 MHz, DMSO) δ 9.85 (s, 1H), 9.60 (s, 1H), 7.02 (t, J=6.2 Hz, 1H), 4.71 (q, J=9.1 Hz, 2H), 3.57 (d, J=6.2 Hz, 2H), 1.38 (s, 9H). ¹⁹F NMR (377 MHz, DMSO) δ −72.82.

2,2,2-Trifluoroethyl 2-glycylhydrazine-1-carboxylate hydrochloride (6): To a solution of compound 5 (7.5 g, 23.79 mmol) in trifluoroethanol (60 mL) was added trimethylsilyl chloride (11.25 mL) slowly at 0 °C, and the resulting solution was stirred for 2 h at RT under an inert condition. After completion of the reaction, as indicated by TLC, solvent was removed under reduced pressure to yield compound 6 as white solid (5.38 g, 90%). ¹H NMR (400 MHz, DMSO) δ 10.52 (s, 1H), 9.86 (s, 1H), 8.33 (s, 3H), 4.74 (q, J=9.0 Hz, 2H), 3.62 (s, 2H). ¹³C NMR (101 MHz, DMSO) δ 166.05, 154.33, 127.73, 124.97, 122.21, 119.45, 60.84, 60.48, 60.14, 59.79, 38.90. ¹⁹F NMR (377 MHz, DMSO) δ −72.83. HRMS (ESI) calculated for C₅H₉F₃N₃O₃ 216.0591, found 216.0587.

### Synthesis of 2-(7-(2-aminoacetamido)-2-oxo-2H-chromen-4-yl) acetic acid

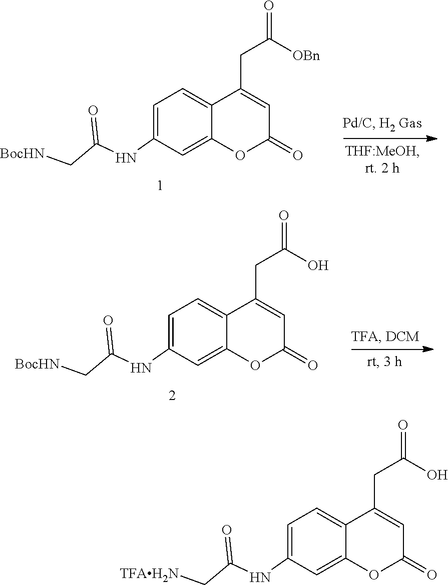

Benzyl 2-(7-(2-((tert-butoxycarbonyl)amino)acetamido)-2-oxo-2H-chromen-4-yl) acetate (1) was synthesized.

2-(7-(2-((tert-butoxycarbonyl)amino)acetamido)-2-oxo-2H-chromen-4-yl) acetic acid (2): To a stirred solution of 1 (2.0 g, 3.43 mmol) in THF:Methanol (1:1, 20 mL) was added 10% Pd/C (160 mg). The reaction flask was sealed and the air inside was removed under the Schlenk line. A hydrogen balloon was then attached to the reaction flask and the reaction mixture was stirred at room temperature for 2 h. After the completion of deprotection, the reaction mixture was filtered with a pad of Celite. The filtrate was concentrated in vacuo and the residue was used for the next step without further purification.

2-(7-(2-aminoacetamido)-2-oxo-2H-chromen-4-yl)acetic acid: To a stirred solution of 2 (1.50 g, 3.98 mmol) in anhydrous DCM (10 mL) at 0 °C was added TFA (3 mL). The reaction mixture was stirred at room temperature for 3 h. After completion of the reaction, remove the solvent in vacuo and wash with hexane to afford SR-D-35 as a white solid (1.0 g, 90%). 1H NMR (400 MHz, DMSO) δ 7.80 (t, J=5.3 Hz, 1H), 7.72 (d, J=8.7 Hz, 1H), 7.44 (d, J=8.8 Hz, 1H), 6.41 (s, 1H), 3.89 (s, 2H), 3.86 (s, 2H).

Compounds and Enzymes for UBL-ACA Assays: Glycyl-2-(7-amino-2-oxo-2H-chromen-4-yl)acetic acid (Gly-ACA) was synthesized as described in the Gly-ACA synthesis section above. TCEP and NTCB were purchased from A2B chem LLC, San Diego, CA, USA. Recombinant SENP1 catalytic domain, USP18, UFSP2, USP16, and ATG4B were acquired from commercial providers or expressed in-house using standard recombinant expression methods. Recombinant UCHL1 was obtained from Sino Biological. Recombinant UCHL3, USP5, USP15, and SENP8 were obtained from UBP Bio or equivalent commercial sources. Recombinant USP2, USP7, and USP9X were obtained from R&D Systems, Medchem Express, or Sino Biological as applicable. Recombinant USP21 was acquired from a commercial provider. N-Ethylmaleimide (NEM) was purchased from Sigma-Aldrich. Ethylenediaminetetraacetic acid (EDTA) was purchased from a standard chemical supplier.

Expression and Purification of FLAG-UBL-GxC-6×His Fusion Proteins: Eleven FLAG-UBL-GxC-6×His expression constructs were generated during previous research, where x denotes the position of the terminal glycine in each UBL and UBL types include SUMO1, SUMO2, SUMO3, SUMO4, URM1, UFM1, NEDD8, ISG15, GABARAP, GABARAPL2, and MNSFβ. Since ISG15, SUMO1, SUMO2, SUMO3, SUMO4, and MNSFβ natively contain a cysteine residue that may undergo the ACPL reaction, this cysteine was mutated to alanine in all six proteins to ensure site-specific conjugation at the C-terminal cysteine only. All eleven FLAG-UBL-GxC-6×His proteins were recombinantly expressed in E. coli BL21(DE3) cells following the protocols described herein. Briefly, an overnight culture of E. coli BL21(DE3) cells harboring the appropriate pETDuet-1 expression vector was inoculated (1:100 dilution) into a 2×YT medium containing 100 μg/mL ampicillin. Cells were grown at 37 °C until OD600 reached 0.6-0.9, and 1 mM IPTG was added to induce protein expression. The cells were grown in a shaker at 37 °C for 8 h or 18 °C for 16 h and collected by centrifugation (5,000 rpm, 30 min, 4 °C). All proteins were purified to above 90% purity using Ni²⁺-NTA affinity chromatography. Purified proteins were analyzed by SDS-PAGE and subjected to electrospray ionization mass spectrometry (ESI-MS) to confirm their intact chemical compositions, with molecular weights matching theoretical values. All purified proteins were concentrated using 3 kDa MWCO Amicon ultracentrifugation filter devices (Millipore Sigma) and concentrations were measured using a NanoDrop spectrophotometer (ThermoFisher). All proteins were aliquoted, lyophilized, and stored at −80 °C.

ACPL Reaction for Synthesis of Ub/UBL-ACA Fluorogenic Substrates: In a one-pot reaction, 500 μM FLAG-UBL-GxC-6×His protein was mixed with 1 mM TCEP, 5 mM NTCB, and 500 mM Gly-ACA in 1×PBS buffer. The reaction mixture was incubated overnight at 37 °C. Gly-ACA was well soluble in these reaction conditions, allowing a 500 mM final concentration to be used, in contrast to Gly-AMC which could only achieve a final concentration of approximately 40 mM. A similar reaction was set up to synthesize Ub-ACA using Ub1-75-Cys-6×His following the UbACA synthesis protocol described herein. All Ub/UBL-ACA products were purified using FPLC. Unreacted proteins containing the C-terminal 6×His tag were then removed by incubation with Ni-NTA resins, and the flow-throughs were collected. The purified probes were analyzed by SDS-PAGE and subjected to ESI-MS. All twelve Ub/UBL-ACA products had determined molecular weights that matched closely with their theoretical values, with a deviation of ≤0.3 Da, confirming their successful synthesis. Using Ub-ACA as an example, the product yield was determined as 26%. To assess whether ACA conjugation disrupts protein folding, circular dichroism (CD) spectroscopy was performed on Ub-ACA using a standard CD spectrophotometer. The CD spectrum of Ub-ACA closely resembled that of native ubiquitin, indicating that the overall protein fold is preserved following ACA conjugation.

Fluorescence Activity Assay for Recombinant DUBs and ULPs Using Ub/UBL-ACA Substrates: All fluorescence activity assays with recombinant DUBs and ULPs were performed in a 96-well plate format. The assay buffer comprised 50 mM Tris, 100 mM NaCl, 0.5 mM EDTA, 1 mM DTT, and 0.1% BSA, pH 7.6. For experiments with USP family enzymes (USP2, USP5, USP7, USP9X, USP15, USP21, USP16, USP18), UCH family enzymes (UCHL1, UCHL3, UCHL5), SENP8, and UFSP2, the enzyme concentration was 50 nM. For SENP1, the enzyme concentration was 100 pM. For ATG4B, the enzyme concentration was 20 nM. All Ub/UBL-ACA substrates were used at 400 nM concentration. Enzymatic cleavage was monitored in real-time by measuring the time-dependent increase in fluorescence resulting from the release of the fluorescent ACA moiety using a fluorescence plate reader (Excitation: 360 nm; Emission: 460 nm). For cross-reactivity control experiments, noncognate enzyme-substrate pairs were evaluated under the same conditions to confirm substrate specificity. All assays were performed at room temperature unless otherwise indicated.

Cell Lysate Profiling of DUB and ULP Activities Using Ub/UBL-ACA Probes: HEK293T cells were cultured in DMEM (Gibco) supplemented with 10% fetal bovine serum (FBS, Gibco) and 5% penicillin-streptomycin (Pen-Strep, Sigma; Pen: 10,000 units/Strep: 10 mg/mL) following the cell culture protocols described herein. The cells were grown in T75 flasks in a 37 °C, 5% CO2 incubator until they reached approximately 90% confluency. The cells were collected in 1×PBS buffer and lysed using Pierce (ThermoFisher) IP protease-free cell lysis buffer as described herein. The resulting supernatant was collected, and the protein concentration was measured by the BCA assay. For the cell lysate profiling assay, cell lysates with an overall protein load of 100 μg were incubated separately with each of the twelve Ub/UBL-ACA probes at 400 nM concentration at 30 °C. Fluorescent release of ACA was monitored continuously for 90 minutes using a fluorescence plate reader (Excitation: 360 nm; Emission: 460 nm).

NEM and EDTA Inhibitor ControlAssays: To confirm that the enzymatic hydrolysis of Ub/UBL-ACA substrates in cell lysates is mediated by cysteine-dependent proteases, inhibitor control experiments were performed. For the NEM quenching assay, HEK293T cell lysates were pretreated with 1 mM N-ethylmaleimide (NEM), a broad-spectrum cysteine protease inhibitor, for a sufficient incubation period to allow covalent modification of active-site cysteine residues prior to the addition of the Ub/UBL-ACA substrate. For the EDTA control assay, HEK293T cell lysates were pretreated with EDTA to sequester divalent metal ions from metalloenzymes. The NEM-pretreated and EDTA-pretreated cell lysates were then incubated with 400 nM of the appropriate Ub/UBL-ACA substrate at 30 °C under the same conditions as described above, and ACA release was monitored by fluorescence plate reader. NEM treatment was expected to completely inhibit cysteine-dependent protease activities, while EDTA treatment served to evaluate the contribution of metalloprotease-mediated hydrolysis.

All of the methods disclosed and claimed herein can be made and executed without undue experimentation in light of the present disclosure. While the compositions and methods of this invention have been described in terms of preferred embodiments or aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. An enzymatic probe of formula I: 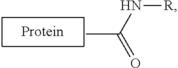
wherein the protein is ubiquitin, a ubiquitin-like protein, or a variant thereof, and wherein R is a hydrazinecarboxylate ester.

2. The enzymatic probe of claim 1, wherein the hydrazinecarboxylate is selected from the group consisting of methyl hydrazinecarboxylate, ethyl hydrazinecarboxylate, 2,2,2-trifluoroethyl-2-glycylhydrazine-1-carboxylate, vinyl hydrazinecarboxylate, allyl hydrazinecarboxylate, and phenyl hydrazinecarboxylate.

3. The enzymatic probe of claim 1, wherein:
a) the ubiquitin-like protein is selected from the group consisting of SUMO1, SUMO2, SUMO3, SUMO4, URM1, UFM1, UFM2, NEDD8, ISG15, FAT10, GABARAP, GABARAPL1, GABARAPL2, MAP1LC3A, MAP1LC3B, MAP1LC3C, ATG12, UBL5, MNSFβ, TUBL, BUBL1, FUBI, and NUB1L;
b) said variant comprises a C-terminal deletion of at least one amino acid;
c) said variant comprises a deletion of a C-terminal glycine, or
d) said variant is ubiquitin₁₋₇₅ or ubiquitin₁₋₇₄.

4. The enzymatic probe of claim 1, further comprising a detectable label.

5. The enzymatic probe of claim 4, wherein the detectable label is selected from the group consisting of a protein tag, a Flag tag, a hemagglutinin A tag, a myc tag, a polyhistidine tag, a CBP tag, a GST tag, an HBH tag, a MBP tag, a S tag, a Sumo tag, a V5 tag, biotin, concanavalin A, a fluorescent label, a chromophore, an avi-tag, and a radioactive label.

6. The enzymatic probe of claim 1, wherein the protein is further defined as an azapeptide.

7. A kit comprising the enzymatic probe of claim 1.

8. The kit of claim 7 comprising at least 2 or at least 3 enzymatic probes of formula I.

9. A method for determining enzymatic activity comprising:
a) contacting a sample comprising at least one enzyme with the enzymatic probe of claim 1; and
b) detecting formation of a covalent complex comprising said enzyme and said enzymatic probe,
wherein the formation of said covalent complex indicates the presence of enzymatic activity.

10. The method of claim 9, wherein the enzyme is a cysteine enzyme.

11. The method of claim 10, wherein the cysteine enzyme is selected from the group consisting of a USP family enzyme, a UCH family enzyme, a Josephin family enzyme, an OTU family enzyme, a ZUFSP family enzyme, an E1 enzyme, an E2 enzyme, and a cysteine-containing E3 enzyme.

12. The method of claim 10, wherein the cysteine enzyme is selected from the group consisting of USP5, USP2, USP7, USP8, USP10, USP14, USP15, USP16, USP18, USP25, UFSP2, CYLD, UCHL1, UCHL3, UCHL5, Ataxin-3, JOSD2, OTUB1, OTUB2, OTULIN, OTUD6B, Cezanne-OTUD7B, OTUD2, OTUD7C, UBE1, UBE2E2, Parkin, UBE3A, ITCH-E3A, USP9X, and USP21.

13. The method of claim 9, wherein the sample comprises a plurality of enzymes.

14. The method of claim 13 comprising contacting the sample with at least 2 or at least 3 enzymatic probes of formula I.

15. The method of claim 9, wherein the hydrazinecarboxylate is selected from the group consisting of methyl hydrazinecarboxylate, ethyl hydrazinecarboxylate, 2,2,2-trifluoroethyl-2-glycylhydrazine-1-carboxylate, vinyl hydrazinecarboxylate, allyl hydrazinecarboxylate, and phenyl hydrazinecarboxylate.

16. The method of claim 9, wherein:
a) the ubiquitin-like protein is selected from the group consisting of SUMO1, SUMO2, SUMO3, SUMO4, URM1, UFM1, UFM2, NEDD8, ISG15, FAT10, GABARAP, GABARAPL1, GABARAPL2, MAP1LC3A, MAP1LC3B, MAP1LC3C, ATG12, UBL5, MNSFβ, TUBL, BUBL1, FUBI, and NUB1L;
b) said variant comprises a C-terminal deletion of at least one amino acid;
c) said variant comprises a deletion of a C-terminal glycine, or
d) said variant is ubiquitin₁₋₇₅ or ubiquitin₁₋₇₄.

17. The method of claim 9, wherein the enzymatic probe further comprises a detectable label.

18. The method of claim 17, wherein the detectable label is selected from the group consisting of a protein tag, a Flag tag, a hemagglutinin A tag, a myc tag, a polyhistidine tag, a CBP tag, a GST tag, an HBH tag, a MBP tag, a S tag, a Sumo tag, a V5 tag, biotin, concanavalin A, a fluorescent label, a chromophore, an avi-tag, and a radioactive label.

19. The method of claim 9, wherein the protein is further defined as an azapeptide.

20. An enzymatic fluorogenic substrate of formula II: 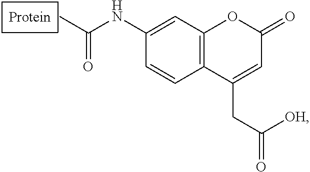 [SMILES: CC(=O)Nc1ccc2c(CC(=O)O)cc(=O)oc2c1]
wherein the protein is ubiquitin, a ubiquitin-like protein, or a variant thereof.

21. The enzymatic fluorogenic substrate of claim 20, wherein:
a) the ubiquitin-like protein is selected from the group consisting of SUMO1, SUMO2, SUMO3, SUMO4, URM1, UFM1, UFM2, NEDD8, ISG15, FAT10, GABARAP, GABARAPL1, GABARAPL2, MAP1LC3A, MAP1LC3B, MAP1LC3C, ATG12, UBL5, MNSFβ, TUBL, BUBL1, FUBI, and NUB1L;
b) said variant comprises a C-terminal deletion of at least one amino acid;
c) said variant comprises a deletion of a C-terminal glycine, or
d) said variant is ubiquitin₁₋₇₅ or ubiquitin₁₋₇₄.

22. The enzymatic fluorogenic substrate of claim 20, wherein the protein is further defined as an amide conjugate.

23. The enzymatic fluorogenic substrate of claim 20, further comprising a detectable label.

24. The enzymatic fluorogenic substrate of claim 23, wherein the detectable label is selected from the group consisting of a protein tag, a Flag tag, a hemagglutinin A tag, a myc tag, a polyhistidine tag, a CBP tag, a GST tag, an HBH tag, a MBP tag, a S tag, a Sumo tag, a V5 tag, biotin, concanavalin A, a fluorescent label, a chromophore, an avi-tag, and a radioactive label.

25. The enzymatic fluorogenic substrate of claim 20, wherein the ubiquitin, the ubiquitin-like protein, or the variant thereof comprises a C-terminal glycine-to-cysteine mutation.

26. A kit comprising the enzymatic fluorogenic substrate of claim 20.

27. The kit of claim 26, further comprising at least two enzymatic fluorogenic substrates, wherein each of the enzymatic fluorogenic substrates comprises a different ubiquitin-like protein or a variant thereof.

28. A method of determining enzymatic activity comprising:
a) contacting a sample comprising at least one enzyme with the enzymatic fluorogenic substrate of claim 20; and
b) detecting hydrolysis of said enzymatic fluorogenic substrate,
wherein hydrolysis of said enzymatic fluorogenic substrate indicates the presence of enzymatic activity.

29. The method of claim 28, wherein said detecting hydrolysis comprises detecting a fluorescent signal or detecting release of the detectable label.

30. The method of claim 28, wherein the enzyme is a deubiquitinase or deubiquitinase-like enzyme.

31. The method of claim 30, wherein the deubiquitinase or the deubiquitinase-like enzyme is selected from the group consisting of a USP family enzyme, a UCH family enzyme, a Josephin family enzyme, an OTU family enzyme, a ZUFSP family enzyme, a JAMM/MPN+ family enzyme, a MINDY family enzyme, and a metalloenzyme.

32. The method of claim 30, wherein the deubiquitinase or the deubiquitinase-like enzyme is selected from the group consisting of USP5, USP2, USP7, USP8, USP10, USP14, USP15, USP16, USP18, USP25, UFSP2, CYLD, UCHL1, UCHL3, UCHL5, Ataxin-3, JOSD2, OTUB1, OTUB2, OTULIN, OTUD6B, Cezanne-OTUD7B, OTUD2, OTUD7C, SENP1, ATG4B, USP9X, and USP21.
